(19) United States
(12) Patent Application Publication
(10) Pub. No.: US 2009/0035314 A1
(43) Pub. Date: Feb. 5, 2009

(54) Method and pharmaceutical composition for preventing or treating diseases associated with inflammation

(76) Inventors: In-San Kim, Daegu (KR); Seung-Yeon Park, Gyeongbuk (KR); Mi-Yeon Jung, Daegu (KR)

Correspondence Address: BUCHANAN, INGERSOLL & ROONEY PC, POST OFFICE BOX 1404, ALEXANDRIA, VA, 22313-1404, US

(21) Appl. No.: 11/665,195

(22) PCT Filed: Oct. 12, 2005
(86) PCT No.: PCT/KR05/03403

§ 371 Date: Apr. 12, 2007

(30) Foreign Application Priority Data
Oct. 12, 2004 (KR) 10-2004-0081498

(51) Int. Cl. A61K 39/395; A61K 38/02; A61K 38/16; C12Q 1/02; A61P 29/00; A61K 31/7052

(52) U.S. Cl. 424/141.1; 514/2; 514/44; 424/130.1; 514/12; 435/29

(57) ABSTRACT

The present invention relates to a method and pharmaceutical composition for preventing or treating inflammatory diseases. More particularly, the present invention relates to a method for inhibiting lymphocyte adhesion to an endothelial cell, or a method for treating an inflammatory disease, which comprises administering to a subject in need thereof an inhibitor against lymphocyte adhesion to a FEX-2 polypeptide. A pharmaceutical composition comprising the inhibitor against lymphocyte adhesion to a FEX-2 polypeptide, and the use of the inhibitor against lymphocyte adhesion to a FEX-2 polypeptide are also disclosed. Further, a method for screening a medicament for inhibiting lymphocyte adhesion to a FEX-2 polypeptide or a medicament for treating an inflammatory disease, which comprises a step of selecting an inhibitor against lymphocyte adhesion to a FEX-2 polypeptide, is disclosed.

### FIELD OF THE INVENTION

This application claims priority to Korean Patent Application No. 10-2004-81498, filed on Oct. 12, 2004, the contents of which are hereby incorporated by reference.

The present invention relates to a method and pharmaceutical composition for preventing or treating an inflammatory disease. More particularly, the present invention relates to a method for inhibiting lymphocyte adhesion to endothelial cells, or a method for treating an inflammatory disease, which comprises administering to a subject in need thereof an inhibitor against lymphocyte adhesion to a FEX-2 polypeptide. The present invention also relates to a pharmaceutical composition comprising the inhibitor against lymphocyte adhesion to a FEX-2 polypeptide, and the use of the inhibitor against lymphocyte adhesion to a FEX-2 polypeptide. Further, the present invention relates to a method for screening a medicament inhibiting lymphocyte adhesion to an endothelial cell or a medicament for treating inflammatory diseases, which comprises a step of selecting an inhibitor against lymphocyte adhesion to a FEX-2 polypeptide.

### BACKGROUND OF THE INVENTION

Inflammation is referred to as a series of reactions in leukocytes for protecting tissues from pathogenic attacks and removing tissue debris produced by damaged tissues. Leukocytes are classified lymphocytes, monocytes and granulocytes (neutrophils, eosinophils and basophils).

Meanwhile, one of the most important steps for carrying out an inflammation reaction is movement of leukocytes from the circulatory system to an inflammation site or wound site. Such movement of leukocytes occurs via a multi-step process including interactions between leukocytes and endothelial cells in the postcapillary venules. In other words, leukocytes move to a site other than blood vessels through the sequential steps of capture, rolling, firm adhesion and transmigration between adjacent endothelial cells (Muller, W. A. wt al., Lab. Invest. 82:521-533, 2002). After leukocytes are adhered to endothelial cells through the above steps, they move from the cell surfaces to an inflammation site or wound site via intracellular junctions (Harlan J. M., Blood, 65: 513-525, 1985).

However, when such inflammatory reactions are controlled inadequately, various kinds of inflammatory diseases arise. General inflammatory diseases include: rhinitis and paranasal sinusitis, such as infectious rhinitis, allergic rhinitis, chronic rhinitis, acute paranasal sinusitis and chronic sinusitis; tympanitis such as acute suppurative tympanitis and chronic suppurative tympanitis; pneumonia such as bacterial pneumonia, bronchial pneumonia, lobar pneumonia, legionella pneumonia and viral pneumonia; enteritis such as acute or chronic gastritis, infectious enterocolitis, Crohn's disease, idiopathic ulcerative colitis and pseudomembranous colitis; arthritis such as suppurative arthritis, tuberculous arthritis, degenerative arthritis and rheumatoid arthritis; and diabetic ophthalmic disease.

It is known that leukocyte adhesion to vascular endothelial cells is mediated by cell adhesion molecule (CAM), in an inflammatory reaction. Besides the function of the CAM as a mediator for leukocyte-vascular endothelial cell adhesion, such cell adhesion molecule is essential to maintain or initiate specialized tissue structure and function, and is crucial to maintain homeostasis of the human body (Edelman, G. M., Annu. Rev Cell Bio., 2:81-116, 1986; Gumbiner, B. M., Cell, 84:345-357, 1996).

Cell adhesion molecules known to date include cadherin, integrin, selectin and immunoglobulin superfamily cell adhesion molecule (IgCAM) (Humphries, M. J. et al., Trends Cell Biol., 8:78-83, 1998). Among those, selectin is known for a calcium ion-dependent cell membrane-bonded lectin family, which initiates adhesion of leukocytes to platelets or endothelial cells (Lasky, Science 258: 964-969, 1992). Further, selectin is classified the following three types: L-selectin expressed in leukocytes, E-selectin expressed in cytokine active endothelial cells, and P-selectin expressed in thrombin active platelets and endothelial cells. Recently, many attempts have been made to develop an anti-inflammatory agent by using an inhibitor against the activity of a cell adhesion molecule such as selectin, which mediates leukocyte-endothelial cell adhesion and causes inflammation.

Korean Patent Publication No. 2004-0039440 discloses an inhibitor against selectin-mediated inflammation, Korean Patent No. 371784 discloses a humanized antibody reactive specifically to L-selectin, for use in treatment of an inflammatory disease.

Meanwhile, a fas-1 domain is a highly preservative sequence, which is found in secreted and membrane-anchored proteins of various species including mammals, insects, sea urchins, plants, yeasts and bacteria (Kawamoto T. et al., Biochim. Biophys, Acta, 288-292, 1998). Additionally, a fas-1 domain comprises about 110 to 140 amino acids. Particularly, a fas-1 domain comprises two subsets (H1 and H2), which comprise about 10 amino acids with high homogeneity and are highly preservative (Kawamoto, T. et al., Biochim. Biophys. Acta., 288292, 1998). Proteins comprising fas-1 domain include βig-h3, periostin, fasciclin I, sea urchin HLC-2, algal-CAM, and mycobacterium MPB70 (Huber, O. et al., EMBO J., 4212-4222, 1994; Matsumoto, S. et al., J. Immunol., 281-287, 1995; Talceshita, S. et al., Biochem. J., 271-278, 1993; Wang, W. C. et al., J. Biol. Chem., 1448-1455, 1993). Among those proteins, βig-h3, periostin and fasciclin I have four fas-1 domains, while HLC-2 has two fas-1 domains and MPB70 has only one fas-1 domain. Although biological functions of proteins comprising fas-1 domains are not clearly demonstrated, it is reported that several proteins function as cell adhesion molecules. Among such proteins, βig-h3 is reported to mediate cell adhesion in fibroblasts and epithelial cells, periostin is reported to mediate cell adhesion in osteoblasts, and fasciclin I is reported to mediate cell adhesion in nerve cells. (LeBaron, R. G. et al., J. Invest. Dermatol., 844-849, 1995; Horinchi, K. et al., J. Bone Miner. Res., 1239-1249, 1999; Wang, W. C. et al., J. Biol. Chem., 1448-1455, 1993). Additionally, algal-CAM is known to function as a cell adhesion molecule present in embryos of volvox (Huber, O. et al., EMBO J., 4212-4222, 1994).

As described above, although several proteins comprising fas-1 domains are known to function as cell adhesion molecules, all proteins are not cell adhesion molecules that contain fas-1 domains.

### DETAILED DESCRIPTION OF THE INVENTION

### Technical Problem

Therefore, the present inventors have conducted many studies to develop a novel therapeutic agent for treating inflammatory diseases and as a result, found that a FEX-2 polypeptide present in endothelial cells is a novel cell adhesion molecule, which mediates adhesion of lymphocytes. Based on this finding, we have demonstrated that a FEX-2 polypeptide-lymphocyte adhesion inhibitor can inhibit lymphocyte adhesion to endothelial cells, and thus is useful for treating inflammatory disease.

### Technical Solution

Therefore, it is an object of the present invention to provide a method for inhibiting lymphocyte adhesion to an endothelial cell, which comprises administering to a subject in need thereof an inhibitor against lymphocyte adhesion to a FEX-2 polypeptide.

It is another object of the present invention to provide a method for preventing or treating an inflammatory disease, which comprises administering an inhibitor against lymphocyte adhesion to a FEX-2 polypeptide to a subject in need thereof.

It is still another object of the present invention to provide a pharmaceutical composition for inhibiting lymphocyte adhesion to an endothelial cell, which comprises an inhibitor against lymphocyte adhesion to a FEX-2 polypeptide and pharmaceutically acceptable carrier.

It is still another object of the present invention to provide a pharmaceutical composition for preventing or treating an inflammatory disease, which comprises an inhibitor against lymphocyte adhesion to a FEX-2 polypeptide and pharmaceutically acceptable carrier.

It is still another object of the present invention to provide the use of an inhibitor against lymphocyte adhesion to a FEX-2 polypeptide for the preparation of a medicament for inhibiting lymphocyte adhesion to an endothelial cell.

It is still another object of the present invention to provide the use of an inhibitor against lymphocyte adhesion to a FEX-2 polypeptide for the preparation of a medicament for preventing or treating an inflammatory disease.

It is still another object of the present invention to provide a method for screening a medicament inhibiting, lymphocyte adhesion to an endothelial cell, which comprises a step of determining whether a test agent inhibits lymphocyte adhesion to a FEX-2 polypeptide.

It is yet another object of the present invention to provide a method for screening a medicament for preventing or treating an inflammatory disease, which comprises a step of determining whether a test agent inhibits lymphocyte adhesion to a FEX-2 polypeptide.

To achieve the above objects, according to an aspect of the present invention, there is provided a method for inhibiting lymphocyte adhesion to an endothelial cell, which comprises administering to a FEX-2 polypeptide to a subject in need thereof an inhibitor against lymphocyte adhesion. According to another aspect of the present invention, there is provided a method for preventing or treating an inflammatory disease, which comprises administering to a FEX-2 polypeptide to a subject in need thereof an inhibitor against lymphocyte adhesion.

According to still another aspect of the present invention, there is provided a pharmaceutical composition for inhibiting lymphocyte adhesion to an endothelial cell, which comprises an inhibitor against lymphocyte adhesion to a FEX-2 polypeptide and pharmaceutically acceptable carrier.

According to still another aspect of the present invention, there is provided a pharmaceutical composition for preventing or treating inflammatory diseases, which comprises an inhibitor against lymphocyte adhesion to a FEX-2 polypeptide and pharmaceutically acceptable carrier.

According to still another aspect of the present invention, there is provided the use of an inhibitor against lymphocyte adhesion to a FEX-2 polypeptide for the preparation of a medicament for inhibiting lymphocyte adhesion to an endothelial cell.

According to still another aspect of the present invention, there is provided the use of an inhibitor against lymphocyte adhesion to a FEX-2 polypeptide for the preparation of a medicament for preventing or treating an inflammatory disease.

According to still another aspect of the present invention, there is provided a method for screening a medicament inhibiting lymphocyte adhesion to an endothelial cell, which comprises a step of determining whether a test agent inhibits lymphocyte adhesion to a FEX-2 polypeptide.

According to yet another aspect of the present invention there is provided a method for screening a medicament for preventing or treating an inflammatory disease, which comprises a step of determining whether a test agent inhibits lymphocyte adhesion to a FEX-2 polypeptide.

Hereinafter, the present invention will be explained in more detail.

Unless otherwise stated, all technical and scientific terms used herein have the same meanings as commonly understood by those ordinary skilled in the art. The following references provide one skilled in the art with general definitions of various terms and expressions used herein. Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); and Hale & Marham, THE HARPER COLLINS DICTIONARY OF BIOLOGY. In addition, definitions of several technical terms are provided hereinafter to help readers.

As used herein, the term ‘polypeptide’ used interchangeably with the terms ‘polypeptides’ and ‘protein(s)’ is referred to a polymer of amino acid residues, typically as found in proteins in nature.

As used herein, a FEX-2 polypeptide may be derived from a mammal, preferably from any one selected from the group consisting of human, rat and mouse. More preferably, a FEX-2 polypeptide is the human FEX-2 polypeptide represented by SEQ ID No:1 or mouse FEX-2 polypeptide represented by SEQ ID No:9. Most preferably, a FEX-2 polypeptide is the human FEX-2 polypeptide represented by SEQ ID No:1.

The above-mentioned FEX-2 polypeptide is gene cloned in a nucleotide database disclosed by the present inventors. It is experimentally demonstrated that a FEX-2 polypeptide is present in vascular endothelial cells and functions to mediate lymphocyte adhesion to vascular endothelial cells.

More particularly, based on the fact that proteins comprising fas-1 domains are found in cell adhesion molecules, the present inventors searched a partial human cDNAs comprising fas-1 domains from a known nucleotide database. Among the searched sequences, three cDNA sequences, whose characteristics are not yet identified were selected. Based on the cDNA sequences, primers were designed, and RT-PCR (reverse-transcription PCR) and 5′ RACE PCR (rapid amplification of cDNA ends) were performed using the total RNA extracted from the human spleen, as a template, and the primers designed as described above. As a result, we cloned a novel human gene comprising fas-1 domains (see Example 1).

The gene synthesized by the present inventors as described above has seven fas-1 domains, twenty-three EGF-like domains, one X-link domain and one transmembrane domain (see FIG. 1). According to the above-described domain structure, the gene was designated as FEX-2 and the gene sequence was registered in GeneBank (AY311388). The human FEX-2 has an amino acid sequence represented by SEQ ID NO:1.

We examined FEX-2 for its expression on a cell surface, in order to determine whether the recombinant FEX-2 protein produced according to the present invention functions as a cell adhesion molecule.

To perform this, we constructed a recombinant vector comprising a human FEX-2 gene. Then, L cells (which are mouse fibroblasts) were transfected with the recombinant vector, and the transfectant was designated as L/FEX-2 (see Example 1). Next, polyclonal antibodies and a monoclonal antibody to the human FEX-2 individually generated. The antibodies were tested by the immunoblotting method to determine whether they can detect the expression of a FEX-2 in the human spleen tissues and L/FEX-2 cells (see Example 2). As a result, it could be seen that the FEX-2 antibodies according to the present invention can specifically detect the FEX-2 protein expressed in the human spleen tissues and L/FEX-2 cells (see FIG. 2).

Thus, we carried out FACS analysis and surface biotinylation assay by using the antibody specific to FEX-2 in order to determine whether FEX-2 is expressed on the L/FEX-2 cell surfaces (see Example 3). As a result, it could be seen that FEX-2 is expressed on the L/FEX-2 cell surfaces (see FIGS. 3 and 4).

Additionally, in order to determine which tissue is applied to the expression of FEX-2, we performed immunohistochemical staining by using the polyclonal human FEX-2 antibody to test the expression of FEX-2 in various human tissues (see Example 4). As a result, it could be seen that FEX-2 is expressed in sinusoidal endothelial cells of the human spleen (see FIG. 5). Also, FEX-2 is expressed in sinusoidal endothelial cells of the liver and lymph nodes (not shown). According to the above results, we estimated that FEX-2 can be expressed in vascular endothelial cells and interact with cells present in the blood.

Then, we examined lymphocyte adhesion to the L/FEX-2 cells, transfected to realize the expression of FEX-2 protein (see Example 5). As a result, it could be seen that a great number of lymphocytes are adhered to the L/FEX-2 cell surfaces compared to the cells used as a control (see FIGS. 6 and 7).

Additionally, we examined lymphocyte adhesion to the L/FEX-2 in the presence of an anti-FEX-2 antibody in order to determine whether the lymphocyte adhesion to the L/FEX-2 cell is caused by FEX-2 protein (see Example 6). As a result, it could be seen that lymphocyte adhesion to the L/FEX-2 cell is inhibited specifically by the anti-FEX-2 antibody (see FIG. 8). Finally, according to the above results, we concluded that FEX-2 functions as a cell adhesion molecule mediating lymphocyte adhesion.

Further, we identified cell, surface receptors to FEX-2 in order to characterize FEX-2 as a cell adhesion molecule in more detail.

To achieve this, according to one embodiment of the present invention, the effect of manganese, magnesium and calcium ions upon lymphocyte adhesion to FEX-2 was determined (see Example 7). As a result, it could be seen that lymphocyte adhesion to FEX-2 is enhanced by manganese ions at the highest degree, and by magnesium ions at the second highest degree. However, calcium ions cannot enhance lymphocyte adhesion to FEX-2 (see FIG. 9). It could be seen from the above results that a cell surface receptor to FEX-2 requires the above divalent cations for the interaction with a ligand. Such a characteristic of the cell surface receptor to FEX-2 is the same as that of the integrin receptor that is bounded to a ligand in a cell adhesion mechanism.

Then, we identified integrin receptors mediating lymphocyte adhesion to FEX-2 (see Example 7). As a result, it could be seen that αLβ2 integrin and αMβ2 integrin interact with FEX-2, and thus participate in the lymphocyte adhesion (see FIG. 10).

From the above results, we have demonstrated for the first time that FEX-2 is present in vascular endothelial cells and has the activity of mediating lymphocyte adhesion to vascular endothelial cells through the interaction between FEX-2 and αLβ2 integrin or αMβ2 integrin of lymphocytes.

Further, we have studied to determine which segment of FEX-2 is related directly with lymphocyte adhesion, and tested whether a polypeptide comprising a segment of FEX-2 related with lymphocyte adhesion can be used as an inhibitor against lymphocyte adhesion to FEX-2.

To perform this, according to another embodiment of the present invention, we divided FEX-2 protein into four subunits and prepared recombinant protein for each subunit (see FIG. 11). Next, we pre-cultured lymphocytes with the subunits, and cultured L/FEX-2 cells expressing FEX-2 with the lymphocytes added thereto.

Then, we determined a degree of lymphocyte adhesion to FEX-2 (see Example <8-1>). As a result, it could be seen that lymphocyte adhesion to FEX-2 is highly inhibited when lymphocytes are pre-cultured with the subunits and are added to L/FEX-2 cells (see FIG. 12).

Further, we divided the third subunit of FEX-2 protein, i.e. Nus-U3 into three segments, and prepared a polypeptide comprising the third EGF-like repeating domain of FEX-2 (Nus-EGF3) and polypeptides each comprising the fifth fas-1 domain and the sixth fas-1 domain (Nus-Fas5 and Nus-Fas6, respectively) (see FIG. 13). Next, lymphocytes were pre-cultured with the polypeptides and were added to L/FEX-2 cells to examine a degree of lymphocyte adhesion to FEX-2 (see Example 8). As a result, when Nus-fas5 and Nus-fas6 polypeptides comprising a fas-1 domain were pre-cultured with lymphocytes, it was possible to inhibit lymphocyte adhesion to L/FEX-2 cells. However, it was not possible to inhibit lymphocyte adhesion in the case of a polypeptide comprising an EGF-like repeating domain (see FIG. 14).

According to still another embodiment of the present invention, we examined a degree of lymphocyte adhesion to FEX-2 protein by pre-culturing lymphocytes with various concentrations of the polypeptide comprising fas-1 domains, and adding the lymphocytes to L/FEX-2 cells (see Example 9). It was shown that inhibition against lymphocyte adhesion to FEX-2 increases as the concentration of the polypeptide increases (see FIG. 15).

Thus, it could be seen from the above results that when lymphocytes are pre-cultured with the polypeptide comprising fas-1 domains, the fas-1 domains are adhered to lymphocytes and serve as competitors against the FEX-2 protein expressed by the L/FEX-2 cells. Therefore, it was demonstrated that fas-1 domains of FEX-2 protein are adhered to lymphocytes, and a polypeptide comprising fas-1 domains is an inhibitor against lymphocyte adhesion to FEX-2 protein.

Further, we examined whether polypeptides comprising seven fas-1 domains present in the human FEX-2 protein and polypeptides comprising fas-1 domains present in other proteins than the human FEX-2 protein, i.e. M. tuberculosis proteins mpt83 and mpt70 can inhibit lymphocyte adhesion to FEX-2 protein (see Example 10). As a result, it could be seen that all of the polypeptides comprising seven fas-1 domains present in FEX-2 protein have the effect of inhibiting lymphocyte adhesion to FEX-2 (FIG. 16). Further, the polypeptides comprising fas-1 domains present in M. tuberculosis proteins mpt83 and mpt70 have the effect of inhibiting lymphocyte adhesion to FEX-2, wherein the lymphocyte adhesion-inhibiting activity depends on the concentration of a polypeptide (see FIG. 17).

As described above, according to the present invention, it was demonstrated for the first time that FEX-2 has the activity of mediating lymphocyte adhesion to vascular endothelial cells through the interaction between FEX-2 and αLβ2 integrin or αMβ2 integrin of lymphocytes. Further, it was shown that lymphocyte adhesion to endothelial cells is inhibited by an inhibitor against lymphocyte adhesion to FEX-2 polypeptide.

Therefore, the present invention provides a method for inhibiting lymphocyte adhesion to endothelial cells, which comprises administering to a subject in need thereof an inhibitor against lymphocyte adhesion to a FEX-2 polypeptide. Such lymphocyte adhesion to endothelial cells is characterized in that it is mediated by cell adhesion molecules comprising fas-1 domains, preferably by a FEX-2 polypeptide.

Meanwhile, an inflammatory reaction occurs when lymphocytes adhere to vascular endothelial cells and then move to an inflammatory site. Herein, if lymphocyte adhesion to a cell adhesion molecule present in vascular endothelial cells is inhibited, it is possible to inhibit the movement of lymphocytes to an inflammatory site, and thus to inhibit an inflammatory reaction (Ulbrich, H. et al., Trends Pharmacol. Sci. 24:640-647, 2003; Harlan, J. M. et al., Crit. Care Med. 30(5 Suppl):S214-219, 2002; van Assche, G. et al., Inflamm. Bowel Dis. 8:291-300, 2002). Accordingly, inhibition against lymphocyte adhesion to FEX-2, which is a cell adhesion molecule present in vascular endothelial cells, results in inhibition against the movement of lymphocyte to an inflammatory site and inflammatory reaction.

Therefore, the present invention provides a method for preventing or treating an inflammatory disease, which comprises administering to a subject in need thereof an inhibitor against lymphocyte adhesion to a FEX-2 polypeptide.

As used herein, the term “subject” means an animal, particularly a mammal, The subject may be a cell, tissue or organ derived from an animal.

As used herein, the term “an inhibitor against lymphocyte adhesion to a FEX-2 polypeptide” is referred to as a compound that is bonded with a FEX-2 polypeptide to inhibit lymphocyte adhesion, or bounded with lymphocyte to inhibit the lymphocyte adhesion to FEX-2, or a compound that inhibits expression of gene encoding FEX-2 protein.

More particularly, the compound that inhibits FEX-2 protein-lymphocyte adhesion includes a peptide, polypeptide, protein, peptide mimic, compound and a biological agent, but is not limited to.

Preferably, the compound that is bounded with FEX-2 to inhibit lymphocyte from being bounded with FEX-2 includes an anti-FEX-2 antibody. Additionally, the compound that is bounded with lymphocyte to inhibit the lymphocyte from being bonded with FEX-2 includes a polypeptide comprising fas-1 domains.

Particularly, the polypeptide comprising fas-1 domains may be derived from a mammal, preferably from any one selected from the group consisting of a human being, rat and mouse. The polypeptide comprising fas-1 domains according to the present invention can utilize fas-1 domains derived from all kinds of proteins known to contain fas-1 domains. Therefore, the fas-1 domain according to the present invention may be a fas-1 domain that can be searched out from a protein sequence database known to one skilled in the art (for example, NCBI Entrez (http://www.ncbi.nlm.nih.gov/Entrez/), EMBL-EBI (http://www.ebi.ac.uk/) or SMART (http://smart.embl-heidelberz.de/).

Preferably, a polypeptide used in the present invention may be comprising fas-1 domains derived from a protein selected from the group consisting of FEX-2, mpt70, mpt83, βig-h3, periostin and FEX-1. More preferably, a polypeptide used in the present invention may be a polypeptide comprising fas-1 domains of human FEX-2, represented by SEQ ID NO: 1 to SEQ ID NO: 12; a polypeptide comprising fas-1 domains derived from M. tuberculosis proteins mpt83 and mpt70, represented by SEQ ID NO: 13 and SEQ ID NO: 14; a polypeptide comprising fas-1 domains derived from mouse FEX-2, represented by SEQ ID NO: 15 to SEQ ID NO: 21; a polypeptide comprising fas-1 domains derived from rat FEX-2, represented by SEQ ID NO: 22 to SEQ ID NO: 25; a polypeptide comprising fas-1 domains derived from human βig-h3 represented by SEQ ID NO: 26 to SEQ ID NO: 29; a polypeptide comprising fas-1 domains derived from mouse βig-h3, represented by SEQ ID NO: 30 to SEQ ID NO: 33; a polypeptide comprising fas-1 domains derived from rat βig-h3, represented by SEQ ID NO: 34 and SEQ ID NO: 35; a polypeptide comprising fas-1 domains derived from human periostin, represented by SEQ ID NO: 36 to SEQ ID NO: 39; a polypeptide comprising fas-1 domains derived from mouse periostin, represented by SEQ ID NO: 40 to SEQ ID NO: 43; a polypeptide comprising fas-1 domains derived from rat periostin, represented by SEQ ID NO: 44 to SEQ ID NO: 47; a polypeptide comprising fas-1 domains derived from human FEX-1, represented by SEQ ID NO: 48 to SEQ ID NO: 54; a polypeptide comprising fas-1 domains derived from mouse FEX-1, represented by SEQ ID NO: 55 to SEQ ID NO: 60; or a polypeptide comprising fas-1 domains derived from rat FEX-1, represented by SEQ ID NO: 61 to SEQ ID NO: 66. Most preferably, a polypeptide used in the present invention may be a polypeptide comprising fas-1 domains of human FEX-2, represented by SEQ ID NO: 1 to SEQ ID NO: 12; or a polypeptide comprising fas-1 domains derived from M. tuberculosis proteins mpt83 and mpt70, represented by SEQ ID NO: 13 and SEQ ID NO: 14. Additionally, such fas-1 domains derived from fas-1 domain-comprising proteins may be used alone or in combination.

Additionally, the range “polypeptide comprising fas-1 domains” also includes a functional equivalent of fas-1 domain and salts thereof. Herein, the term “functional equivalent” is referred to as a polypeptide showing the substantially same physiological activity as fas-1 domain. In other words, an amino acid sequence as well as a structurally equivalent or similar polypeptide may be used in the present invention, as long as it shows the same physiological activity as the polypeptide according to the present invention. The term “substantially the same physiological activity” means the activity of inhibiting lymphocyte adhesion to a cell adhesion molecule, FEX-2. More particularly, the above term means the activity of inhibiting lymphocyte adhesion to FEX-2 through the interaction with αLβ2 integrin or αMβ2 integrin of lymphocytes.

Additionally, as used herein, the range of functional equivalent includes a polypeptide derivative that maintains the fundamental skeleton and physiological activity of the polypeptide according to the present invention and has a modified chemical structure. For example; the functional equivalent includes a polypeptide having a structure modified so as to change stability, storage stability, volatility or solubility of the polypeptide.

The polypeptide comprising fas-1 domains according to the present invention may be obtained with ease by a chemical synthesis process known to one skilled in the art (Creightion, Proteins; Structures and Molecular Principles, W. H. Freeman and Co., NY, 1983). Typical examples of the process include a liquid or solid phase process, fragment condensation process, F-BOC or a T-MOC chemical process (Chemical Approaches to the Synthesis of Peptides and Proteins, Williams et al., Eds., CRC Press, Boca Raton Fla., 1997; A Practical Approach, Athert on & Sheppard, Eds., IRL Press, Oxford, England, 1989), but are not limited to.

Further, the polypeptide comprising fas-1 domains may be produced by a biotechnological process. In other words, a DNA sequence encoding the polypeptide is provided in a conventional manner. The DNA sequence may be prepared by PCR amplification using adequate primers. In a different method, for example, the DNA sequence may be produced by using an automatic DNA producing instrument known to one skilled in the art (available from Biosearch or Applied Biosystems Co.). Then, the DNA sequence is inserted into a vector comprising at least one expression control sequence (e.g. promotor, enhancer, etc.) that is operatively linked to the DNA sequence to control the expression of the DNA sequence, and the resultant recombinant expression vector is used to transfect a host cell. The transfected cell is cultured in a suitable medium under suitable conditions sufficient to realize expression of the DNA sequence. Then, substantially pure polypeptide encoded by the DNA sequence is obtained from the cultured product. The obtainment step may be performed by a conventional method (e.g. chromatography). Herein, the expression “substantially pure polypeptide” is referred to as the polypeptide according to the present invention, which does not comprise any other proteins derived from the host cell. The following references provide detailed description of a biotechnological process for producing the polypeptide according to the present invention: Maniatis et al., Molecular Cloning; A laboratory Manual, Cold Spring Harbor laboratory, 1982; Sambrook et al., supra; Gene Expression Technology, Method in Enzymology, Genetics and Molecular Biology, Method in Enzymology, Guthrie & Fink (eds.), Academic Press, San Diego, Calif., 1991; and Hitzeman et al., J. Biol. Chem., 255:12073-12080, 1990.

According to the present invention, the anti-FEX-2 antibody may be a polyclonal or a monoclonal antibody. The antibody according to the present invention may be produced via a conventional method known to the field of immunology by using FEX-2 protein as an antigen.

The polyclonal antibody may be produced in a conventional manner from various homoiothermal animals including horses, cows, goats, sheep, dogs, chickens, turkeys, rabbits, mice or rats. In other words, an antigen is injected through an intraperitoneal, intramuscular, intraocular or subcutaneous route to immunize an animal. Immunity against the antigen may be increased by using an adjuvant such as the Freund complete adjuvant or incomplete adjuvant. After the booster immunization, a small sample of blood sera is collected and tested for reactivity to a target antigen. If a titer of the animal reaches the plateau when viewed from the reactivity to the antigen, a large amount of polyclonal immunosera can be obtained through weekly bleeding of the animal or phlebotomy of the animal.

Also, the monoclonal antibody may be produced by a conventional process (Kennettm McKearn, and Bechtbl (eds.), Monoclonal Antibodies, Hybridomas; A New Dimension in Biological Analyses, Plenum Press, 1980). The monoclonal antibody may be produced by a process comprising the steps of: immunizing an animal by using FEX-2 protein as an immunogen; generating hybridoma cells by fusing the spleen cells of the immunized animal with myeloma cells; selecting a hybridoma that recognizes FEX-2 protein selectively; culturing the selected hybridoma; and separating an antibody from the hybridoma culture. Additionally, the monoclonal antibody according to the present invention may be produced by injecting the hybridoma generating the anti-FEX-2 antibody that recognizes FEX-2 protein selectively into an animal, and then isolating the antibody from the ascites collected from the animal after the lapse, of a time.

Hybridoma 5G3 producing the monoclonal human FEX-2 antibody, obtained according to one embodiment of the present invention, was deposited in one of the international depository authorities, i.e. the Korean Collection for Type Cultures (KCTC) located within the biological resources center of the Korea Research Institute of Bioscience and Biotechnology (52, Eoeun-dong, Yuseong-ku, Taecheon, Korea) as the Accession No. KCTC-10639BP on May 21, 2004. The antibody deposited as described above may be maintained alive for the total period of the issued patent by keeping it in the KCTC, and may be available for any person or entity for the non-commercial purpose with no limitation according to the provisions of the Deposit Management Law.

In addition, the compound that inhibits expression of genes encoding FEX-2 protein includes a substance that inhibits transcription of the genes or translation of the genes into proteins. Such inhibition against expression of genes includes not only complete termination of gene expression but also reduction of gene expression.

A typical example of the substance that inhibits expression of genes encoding FEX-2 protein is an antisense molecule capable of inhibiting expression of specific intrinsic genes. The actions of the antisense molecule to inhibit the expression of a target gene include: the inhibition of transcription initiation, by the formation of a triple-chain structure; the transcriptional inhibition by the hybrid formation at the site where a local opened loop structure was made by RNA polymerase; the transcription inhibition by the hybrid formation at the RNA being synthesized; splicing inhibition by the hybrid formation at an intron-exon junction; the splicing inhibition by the hybrid formation at a splicosome-forming site; the inhibition of migration from a nucleus to cytoplasm by the hybrid formation with mRNA; and the inhibition of translation initiation by the hybrid formation at a translation initiator-binding site. Such antisense molecules inhibit a process of transcription, splicing or translation, and thus inhibit the expression of a target gene.

The antisense molecule used in the present invention may inhibit the expression of a target gene by any of the above action. Typical antisense molecules include a triple helix forming agent, ribozyme, RNAi or an antisense nucleic acid. The triple helix forming agent is circularized around a double-strand DNA to form a triple helix, thereby inhibiting transcription initiation (Maher et al., Antisense Res. and Dev., 1(3):227, 1991; Helene, C., Anticancer Drug Design, 6(6):569, 1991).

The ribozyme is an enzyme capable of cleavage of a single-strand RNA.

The ribozyme recognizes a specific nucleotide sequence in a target RNA molecule and cleaves the sequence in a site-specific manner, thereby inhibiting the protein expression of the target genes (Cech, J. Amer. Med. Assn., 260:3030, 1998; Sarver et al., Science 247:1222-1225, 1990).

The RNAi (RNA interference) is a method for inhibiting gene expression in a transcription level or post-transcription level by using a hairpin-shape small molecule RNA, which acts in a sequence-specific manner (Mette et al., EMBO J., 19: 5194-5201, 2000). The small molecule RNA used in the RNAi method is a double-strand RNA molecule homologous to the target gene.

Herein, the RNA molecule may be produced by a conventional chemical or enzymatic process. For example, the RNA molecule may be produced by a chemical process disclosed in the art (Verma and Eckstein, Annu. Rev. Biochem. 67, 99-134, 1999). An enzymatic process for producing an RNA molecule by using an RNA polymerase such as a phage T7, T3 or SP6 polymerase is described in (Milligan and Uhlenbeck, Methods Enzymol. 180:51-62, 1989).

The antisense nucleic acid is referred to as a DNA or RNA molecule at least partially complementary to the target mRNA molecule (Weintraub, Scientific American, 262:40, 1990). From the intracellular point of view, the antisense nucleic acid is hybridized with mRNA corresponding thereto to form a double-strand molecule, thereby inhibiting decoding of mRNA of the target gene and protein expression (Marcus-Sakura, Anal. Biochem., 172:289, 1988). Such antisense nucleic acids are particularly useful for the inhibitor against the expression of FEX-2 according to the present invention, Preferably, the antisense nucleic acid may be produced in the form of an oligonucleotide by a suitable method known to one skilled in the art. More particularly, the antisense oligonucleotide may be produced by a chemical process, for example by the chemical phosphoamidite method comprising sulfuration with tetraethylthiuram disulfide in acetonitrile (Tetrahedron Lett., 1991, 32, 30005-30008).

Inflammatory diseases that can be prevented or treated by the method according to the present invention include an inflammatory reaction itself or various diseases caused by inflammatory reactions. Particular non-limiting examples of the inflammatory diseases include: inflammation, inflammatory bowl disease, diabetic ocular disease, peritonitis, osteomyelitis, cellulitis, meningitis, encephalitis, pancreatitis, trauma causing shock, bronchial asthma, rhinitis, sinusitis, otitis media, pneumonia, gastritis, enteritis, cystic fibrosis, apoplexy, bronchitis, bronchiolitis, hepatitis, nephritis, arthritis, gout, spondylitis, Reiter's syndrome, polyarteritis nodosa, hypersensitivity vasculitis, Wegener's granulomatosis, polymyalgia rheumatica, giant cell arteritis, calcium crystal deposition arthropathy, pseudogout, nonarticular rheumatism, bursitis, tenosynovitis, epicondylitis (Tennis elbow), neuropathic joint disease (Charcot's joint), hemarthrosis, Henoch-Schonlein Purpura, hypertrophic osteoarthropathy, multicentric reticulohistiocytoma, scoliosis, hemochromoatosis, sickle cell disease and other hemoglobinopathies, hyperlipoproteinemia, hypogammaglobulinemia, hyperparathyroidism, acromegaly, familial mediterranean fever, Behcet's disease, systemic lupus erythematosus, relapsing fever, psoriasis, multiple sclerosis, septicemia, septic shock, acute respiratory distress syndrome, multiple organ failure, chronic obstructive pulmonary disease, acute lung injury and broncho-pulmonary dysplasia.

According to another aspect of the present invention, the present invention provides a pharmaceutical composition for inhibiting lymphocyte adhesion to endothelial cells, which comprises an inhibitor against lymphocyte adhesion to a FEX-2 polypeptide and pharmaceutically acceptable carrier.

The present invention also provides a pharmaceutical composition for preventing or treating inflammatory disease, which comprises an inhibitor against lymphocyte adhesion to a FEX-2 polypeptide and pharmaceutically acceptable carriers.

As used herein, the term “pharmaceutically acceptable carrier” is referred to as a composition that is physiologically acceptable and does not cause any allergic reactions or similar reactions such as gastrointestinal disorders or vertigos, when administered to the human body. The pharmaceutically acceptable carriers may include, for example, oral carrier, and parenteral administration carrier. The oral carrier may include lactose, starch, cellulose derivatives, magnesium stearate, and stearic acid. Also, the parenteral carrier may include water, suitable oil, saline solution, aqueous glucose and glycol. The composition according to the present invention may further comprise stabilizer and preservative. Suitable stabilizer includes antioxidant, such as sodium bisulfite, sodium sulfite or ascorbic acid. Suitable preservative includes benzalkonium chloride, methyl- or propyl-paraben, and chlorobutanol. For other pharmaceutically acceptable carriers, reference may be made to the following literature (Remington's Pharmaceutical Sciences, 19th ed., Mack Publishing Company, Easton, Pa., 1995).

The pharmaceutical composition according to the present invention may be mixed with the pharmaceutically acceptable carriers as described above and formulated into an adequate form by means of a process known to one skilled in the art. In other words, the pharmaceutical composition according to the present invention may be formulated into various forms for oral or parenteral administration by a conventional process known to one skilled in the art. The formulations for parenteral administration preferably include injection formulations, such as isotonic aqueous solution or suspension formulations. The injection formulations may be prepared by using suitable dispersing or wetting agents, and suspending agents, according to any technique known in the art. For example, formulations for injection may be obtained by dissolving necessary components in a saline or buffer solution. Examples of the formulations for oral administration include powder, granule, tablet, pill and capsule, but are not limited to.

The pharmaceutical composition formulated as described above may be administered in an effective amount through various routes including oral, transdermal, subcutaneous, intravenous and intramuscular routes. The term “effective amount” is referred to as the amount of a compound or extract that shows a preventive or therapeutic effect. The dose of the inventive pharmaceutical composition may be suitably selected according to an administration route, a subject to be administered, and the age, sex, body weight, characteristic and disease condition of the subject. Preferably, the pharmaceutical composition comprising the polypeptide according to the present invention may be administered to an adult at an effective unit dose ranging from about 10 μg to 10 mg, one time or several times per day. However, the effective dose may be varied depending on the severity of diseases.

Also, the present invention provides the use of an inhibitor against lymphocyte adhesion to a FEX-2 polypeptide for the preparation of a medicament for inhibiting lymphocyte adhesion to endothelial cells.

Also, the present invention provides the use of an inhibitor against lymphocyte adhesion to a FEX-2 polypeptide for the preparation of a medicament for preventing or treating inflammatory diseases.

Further, the present invention provides a method for screening a medicament for inhibiting lymphocyte adhesion to a FEX-2 polypeptide.

More particularly, when a test agent functions specifically to the cells expressing FEX-2, the screening method according to the present invention comprises the steps of:

(a) pre-culturing cells expressing a FEX-2 polypeptide with or without a test agent;

(b) adding lymphocytes to the cells pre-cultured with or without the test agent in the step (a) and further culturing them; and

(c) measuring a degree of lymphocyte adhesion to the cells pre-cultured with the test agent, and comparing the measured degree with a degree of lymphocyte adhesion to the cells pre-cultured without the test agent, thereby determining whether the test agent inhibits lymphocyte adhesion.

Additionally, when a test agent functions specifically to lymphocytes, the screening method according to the present invention comprises the steps of:

(a) pre-culturing lymphocytes with or without a test agent;

(b) adding the lymphocytes pre-cultured with or without the test agent in the step (a) to cells expressing FEX-2 polypeptide and further culturing them; and

(c) measuring a degree of lymphocyte adhesion to the cells pre-cultured with the test agent, and comparing the measured degree with a degree of lymphocyte adhesion to the cells pre-cultured without the test agent, thereby determining whether the test agent inhibits lymphocyte adhesion.

Further, the present invention provides a method for screening a medicament for preventing or treating inflammatory diseases.

According to the present invention, the method for screening a medicament for preventing or treating an inflammatory disease further comprises step (d) of administering the test agent determined to inhibit lymphocyte adhesion in the step (c) to an animal suffering from an inflammatory disease to examine a therapeutic effect, in addition to the above steps (a) to (c) of the method for screening a medicament for inhibiting lymphocyte adhesion to a FEX-2 polypeptide.

In the above method, the animal is preferably a non-human animal. Additionally, the term “therapeutic effect” used in describing the step (d) means the effect of alleviating or improving an inflammatory disease and or the effect of inhibiting the progress of inflammatory disease.

As used herein, the term “agent” or “test agent” includes any substances, molecules, elements, compounds, entities or combinations thereof. Particular examples thereof include, but are not limited to, proteins, polypeptides, small organic molecules, polysaccharides and polynucleotides. Also, the test agent may be a natural product, synthetic product, chemical compound or a combination thereof. Unless otherwise indicated, the terms “agent”, “substance” and “compound” may be interchangeable.

The test agents that can be screened or identified by the inventive methods include polypeptides, beta-turn mimetics, polysaccharides, phospholipids, hormones, prostaglandins, steroids, aromatic compounds, heterocyclic compounds, benzodiazepines, oligomeric N-substituted glycines, oligocarbamates, saccharides, fatty acids, purines, pyrimidines, or derivatives, structural analogs or combinations thereof. Some test agents may be synthetic molecules and others natural molecules. The test agent may be available from various sources including libraries of synthetic or natural compounds. A combinatorial library may be produced from various kinds of compounds that can be synthesized in a step-by-step manner. A plurality of compounds in combinatorial libraries may be produced by the ESL (encoded synthetic libraries) method (WO 95/12608, WO-93/06121, WO 94/08051, WO 95/395503 and WO 95/30642). Libraries of natural compounds present in the form of extracts of bacterial, fungal, plant and animal are commercially available or can be obtained in the field. Additionally, known pharmacological agent can be subjected to a directed or random chemical modification, including acylation, alkylation, esterification and amidification, so as to obtain its structural analogs. Also, the test agent may be a naturally occurring protein or a fragment thereof. Such test agents may be obtained from a natural source such as a cell, or tissue lysate. Libraries of a polypeptide preparation may be obtained from cDNA libraries that are produced by a conventional method or are commercially available. The test agent may be a peptide (for example a peptide having about 5-30, preferably about 5-20, more preferably about 7-15 amino acids). The peptide may be a cleaved product of naturally occurring proteins, random peptides or biased random peptides.

The test agent may also be a “nucleic acid”. The nucleic acid test agent may be a naturally occurring nucleic acid, random nucleic acid or biased random nucleic acid. For example, a cleaved product of procaryotic or eukaryotic genomes may be used in a similar manner.

The test agent may also be a small molecule (e.g. a molecule having a molecular weight of about 1,000 or less). As a method for screening an agent for controlling such small molecules, a high throughput assay may be used preferably. As described above, combinatorial libraries of small molecule test agents may be applied to the screening method according to the present invention. Several assay systems are useful for the screening method (Shultz, Bioorg. Med. Chem. Lett., 8:2409-2414, 1998; Weller, Mol. Drivers., 3:61-70, 1997; Fernandes, Curr. Opin. Chem. Biol., 2:597-603, 1998; and Sittampalam, Curr. Opin. Chem. Biol., 1:384-91, 1997).

As used herein, the term “cells expressing FEX-2 polypeptide” is referred to as cells transfected with a vector comprising the genes encoding a FEX-2 polypeptide. Although there is no particular limitation in the cells, it is preferable that the cells have no additional cell adhesion molecules other than a FEX-2 polypeptide. For example, the cells include mouse fibroblasts, L cells. Besides the mouse L cells, it may be used Chinese hamster ovary cells (CHO), mouse sarcoma cells S180 and Drosophila S2 cells.

The vector comprising the genes encoding a FEX-2 polypeptide may be produced with ease by producing cDNA from a known FEX-2 genetic sequence through a known method, and cloning the cDNA to a suitable vector. For example, the expression vector may be pcDNA-Fex2.

Preferably, lymphocytes used in the above method are marked with a fluorescence dye. Any fluorescence dye may be used, as long as it causes lymphocytes to be dyed via diffusion into cell membranes and it can be observed with a fluorescence microscope.

The degree of lymphocyte adhesion to the transfected cells can be measured by counting the number of lymphocytes adhered to the transfected cells with an optical microscope. Otherwise, it can be measured by culturing lymphocytes along with the transfected cells, carrying out lysis of the cells with a cell lysis buffer solution to provide a lysate, and determining the fluorescence of the lysate.

When the number of lymphocytes is counted by using a microscope, lymphocytes are cultured along with the transfected cells, and non-adhered lymphocytes are washed out. Then, the number of lymphocytes is counted by using a microscope at randomly selected 10 positions, and the 10 measurements are averaged.

When the fluorescence of the cell lysate is measured, lymphocytes are cultured along with the transfected cells, and non-adhered lymphocytes are washed out. Then, the cell lysate obtained by adding a cell lysis buffer solution was determined for the amount of fluorescence by using a fluorescence microplate reader.

The foregoing and other objects, features and advantages of the present invention will become more apparent from the following detailed description when taken in conjunction with the accompanying drawings.

### BRIEF DESCRIPTION OF THE DRAWINGS

FIG. 1 is a schematic view showing each domain of the human FEX-2 protein.

FIG. 2 shows the results of Western blot assay for the expression of FEX-2 protein in the human spleen tissue, L/FEX-2 cells and L/Mock cells using the monoclonal human FEX-2 antibody.

FIG. 3 shows the results of FACS (fluorescence activated cell sorter) analysis for the expression of FEX-2 protein on the surface of L/Mock cells and L/FEX-2 cells using the monoclonal human FEX-2 antibody.

FIG. 4 shows the results of surface biotinylation analysis for the expression of FEX-2 protein on the surface of L/FEX-2 cells (−: no addition, +: addition).

FIG. 5 shows the results of immunostaining using the polyclonal human FEX-2 antibody in the human spleen tissue (a: control, b: test sample).

FIG. 6 is a photograph taken by a microscope, which shows a degree of lymphocyte adhesion in L/Mock cells and L/FEX-2 cells (a: L/Mock cells, b: L/FEX-2 cells, arrow heads: lymphocytes).

FIG. 7 is a graph showing a degree of lymphocyte adhesion in L/Mock cells and L/FEX-2 cells.

FIG. 8 is a graph showing inhibition of lymphocyte adhesion to L/FEX-2 cells, induced by the monoclonal human FEX-2 antibody.

FIG. 9 is a graph showing the effects of various divalent cations upon lymphocyte adhesion to L/FEX-2 cells.

FIG. 10 is a graph showing inhibition of lymphocyte adhesion to L/FEX-2 cells, induced by various kinds of inhibition antibodies against integrin:

β1: treated with anti-β1 antibody,

αLβ2: treated with anti-αL antibody,

αMβ2: treated with anti-αM antibody, and

αXβ2: treated with anti-αX antibody.

FIG. 11 is a schematic view of the human FEX-2 protein divided into four subunits.

FIG. 12 is a graph showing inhibition of lymphocyte adhesion to FEX-2 in four subunits of the human FEX-2 protein:

Nus-U1: Nus protein followed by the first subunit of FEX-2 protein,

Nus-U2: Nus protein followed by the second subunit of FEX-2 protein

Nus-U3: Nus protein followed by the third subunit of FEX-2 protein, and

Nus-U4: Nus protein followed by the fourth subunit of FEX-2 protein.

FIG. 13 is a schematic view of the third subunit (Nus-U3) of the human FEX-2 protein, further divided into three segments.

FIG. 14 is a graph showing inhibition of lymphocyte adhesion to FEX-2, induced by polypeptides produced by using Nus-U3 of the human FEX-2 protein, divided into three segments:

Nus-U3: Nus protein followed by the third subunit of FEX-2 protein,

Nus-EGF3: Nus protein followed by the third EGF-like repeating domain of FEX-2 protein,

Nus-Fas5: Nus protein followed by the fifth fas-1 domain of FEX-2 protein, and

Nus-Fas6: Nus protein followed by the sixth fas-1 domain of FEX-2 protein.

FIG. 15 is a graph showing inhibition of lymphocyte adhesion to FEX-2 varying with concentrations of polypeptides, when using the polypeptides obtained from Nus-U3 of the human FEX-2 protein, divided into three segments:

Nus-Unit: Nus protein followed by the third subunit of FEX-2 protein,

Nus-EGF: Nus protein followed by the third EGF-like repeating domain of FEX-2 protein, and

Nus-Fas: Nus protein followed by the fifth fas-1 domain of FEX-2 protein.

FIG. 16 is a graph showing inhibition of lymphocyte adhesion to FEX-2, induced by polypeptides comprising seven fas-1 domains present in FEX-2 protein.

FIG. 17 is a graph showing inhibition of lymphocyte adhesion to FEX-2, induced by polypeptides comprising fas-1 domains present in M. turberculosis proteins mpt83 and mpt70.

### BEST MODE FOR CARRYING OUT THE INVENTION

Hereinafter, the present invention will be described in detail by way of the following examples. However, it is to be understood that these examples are given for illustrative purpose only and are not intended to limit the scope of the present invention.

### Example 1

### Cloning of Human FEX-2 cDNA

<1-1> Construction of Expression Vector

To identify a novel cell adhesion molecule comprising fas-1 domains, sequences comprising fas-1 domains were searched nucleotide database such as Genebank and Celera genomics. After the search, non-characterized partial human cDNA clones (FLJ00012, DZKZp434E0321, CD44-like precursor FELL) were selected. The full-length cDNA sequence was designed from the human spleen tissue, by means of RT-PCR and 5′ RACE PCR.

First, two pairs of primers (sequence Nos. 67 to 70) were designed by using the above three partial cDNA clones derived from the human spleen, comprising fas-1 domains (Table 1). Next, RT-PCR (reverse transcriptase polymerase chain reaction) was performed by using 2 μg of RNA extracted from the human spleen, as a template, and each pair of primers designed as described above, so that two segments of the partial cDNA of a protein comprising fas-1 domains were obtained. The PCR reaction was performed using a PCR system (Expand high fidelity PCR system, Roche) under the following conditions: 2 min at 95° C.; and 30 cycles of, 30 sec at 94° C., 30 sec at 60° C. and 30 sec at 72° C. As a result, amplified products of 3.6 kb and 2.0 kb were obtained. Among the products, 3.6 kb cDNA was digested by restriction enzymes ClaI and SacI (TaKaRa), and then inserted into pBluescript-KS(+) vector (Stratagene) at the sites of the same restriction enzymes by using T4 ligase (Invitrogen), thereby constructing recombinant vector ‘pBS-Fex23’. On the other hand, 2.0 kb cDNA was digested by restriction enzymes SacI and HindIII (TaKaRa), and then inserted into pET-29b vector (Novagen) at the sites of the same restriction enzymes by using T4 ligase (Invitrogen), thereby constructing recombinant vector ‘pET-Fex45’. Then, fusion of both cDNA sequences was performed as follows. First, a fragment obtained by digesting pET-Fex45 with EcoRI was treated with the klenow enzyme to provide a blunt fragment. Next, the blunt fragment was further digested with SacI, and was cloned to the recombinant vector PET-Fex23 at the sites of the same restriction enzymes, thereby constructing recombinant vector ‘pET-Fex2345’.

Then, 5′-end of cDNA comprising fas-1 domains was determined as follows. RNA obtained from the human spleen was amplified with a primer (sequence No. 71) as shown in Table 1 to provide cDNA. An amplified product was obtained by means of 5′ RACE PCR using the cDNA as a template, and primers as shown in Table 1 (sequence Nos 72 and 73) and the adapter primer provided by the 5′ RACE system (5′ RACE system for Rapid Amplification of cDNA Ends version 2.0, Invitrogen), according to the manufacturer's instructions. Then, the amplified product was analyzed in a sequence analyzer (Applied Biosystems AB13700) to determine the 5′-end sequence of FEX-2. A primer was designed by using the 5′-end sequence. RT-PCR (reverse transcriptase polymerase chain reaction) was performed by using 2 μg of RNA extracted from the human spleen, as a template, and each pair of primers (sequence Nos. 74 and 75) designed from the 5′-end sequence as described above, so that 2.5 kb 5′-end cDNA was obtained. The amplified product was cleaved by restriction enzymes EcoRI and ClaI (TaKaRa), and then inserted into pBluescript-KS(+) vector (Stratagene) at the sites of the same restriction enzymes, thereby constructing recombinant vector ‘pBS-Fex1’. Cloning of the complete sequence to the expression vector was performed as follows. First, pET-Fex2345 obtained as described above was cleaved by restriction enzymes KpnI and HindIII, and then inserted into pcDNA3.1(−)/Myc-His vector (Invitrogen) at the sites of the same restriction enzymes, thereby constructing ‘pcDNA-Fex45’. Also, pET-Fex 2345 was further cleaved by BamHI and KpnI, and then inserted into pcDNA-Fex45 at the sites of the same restriction enzymes, thereby constructing ‘pcDNA-Fex2345’. Finally, pBS-Fex1 obtained as described above was digested by EcoRI and ClaI, and then inserted into pcDNA-Fex2345 at the sites of the same restriction enzymes.

The plasmid obtained as described above was analyzed in a sequence analyzer (Applied Biosystems AB13700). The sequence was deposited in the Genebank (Genebank™ accession number AY311388), and the expression vector comprising the complete sequence of FEX-2 was designated as ‘pcDNA-Fex2’.

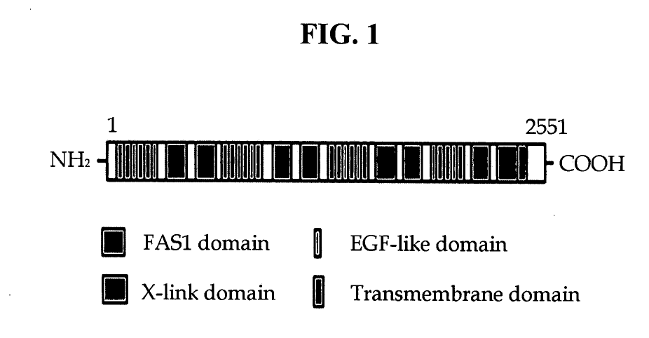

It was shown that the amino acid sequence estimated from the sequence analysis results was substantially the same as scavenger receptor FEEL-2 (Adachi H. et al., J. Biol. Chem. 277:34264-34270, 2002) and encocytic hyaluronan receptor Stabilin-2(Politz O. et al., Biochem J. 362:155-164, 2002). FEX-2 comprised seven fas-1 domains, twenty-three EGF-like domains, one X-link domain and one transmembrane domain (FIG. 1).

TABLE 1

Primers used in cloning of Fex-2

| | | |
|---|---|---|
| Primers | Sequence | Seq. No. |

| | | |
|---|---|---|
| Fex23 sense | 5′-tgc ccc atc gat gtg atg aaa c-3′ | 67 |
| Fex23 antisense | 5′-caa aca aga gct ccc gct gca caa t-3′ | 68 |
| Fex45 sense | 5′-gca gcg gga gct ctt gtt tga cct g-3′ | 69 |
| Fex45 antisense | 5′-ccc gtc can gct tgc aca gtg tcc t-3′ | 70 |
| RACE-GSP-1 antisense | 5′-tcc cag ctt act cag tgg cca ggc-3′ | 71 |
| RACE-GSP-2 antisense | 5′-cag gcc cat cat att tgc aca ctg tag ac-3′ | 72 |
| RACE-GSP-3 antisense | 5′-agt taa ttt ggc agg ggt cca cag gc-3′ | 73 |
| Fex1 sense | 5′-aag gca ggt ctc acc tat ctc ctg g-3′ | 74 |
| Fex1 antisense | 5′-tca caa atg cat gtc ccg ttg c-3′ | 75 |

<1-2> Transformation of L Cells

Mouse fibroblasts, L cells (ATCC CCL-1, obtained from Dr. Dakechi, Dept. of biophysics, Tokyo Univ.) were transfected with the expression vector pcDNA-Fex2 constructed in Example <1-1>. L cells were cultured in a DMEM medium (Dulbecco's modified Eagle's medium) supplemented with 10% heat-inactivated FBS, penicillin G and streptomycin. The mouse L cells are cell lines free from the expression of cadherin (which is a typical-cell adhesion molecule), and thus are widely used for the study of cell adhesion-activities (Nose, A. Cell 54:993-1001, 1988).

The L cells were transfected with the recombinant vector pcDNA-Fex2 by using lipofectamine (Invitrogen) according to the manufacturer's instructions. Next, 48 hours after the transfection, cells were treated with G418 (400 μg/ml) and cultured for 10 to 12 days. During the culture period, individual colonies showing G418-resistance were isolated. The cells transfected as described above were designated as L/FEX-2. As a negative control (L/Mock), cells transfected with the vector pcDNA3.1 (−)/Myc-His comprising no FEX-2 genes were used.

### Example 2

### Production of Polyclonal Antibody and Monoclonal Antibody Against FEX-2

<2-1> Production of Polyclonal Antibody Against Human FEX-2

To produce polyclonal antibodies against human FEX-2, cDNA comprising a sequence corresponding to amino acids 554 to 655 from the complete human FEX-2 amino acid sequence (sequence No. 1) and cDNA comprising a sequence corresponding to amino acids 2188 to 2551 were produced by PCR amplification. The cDNAs were obtained by means of PCR using the pcDNA-Fex2 DNA according to Example <1-1>, as a template, and the following primers (sequence Nos. 76 to 79) (Table 2). The PCR reaction was performed under the following conditions: 2 min at 95° C.; and 25 cycles of, 30 sec at 94° C., 30 sec at 60° C. and 30 sec at 72° C. The amplified product comprising the sequence corresponding to amino acids 554 to 655 was digested by restriction enzymes SalI and HindIII (TaKaRa) and inserted into pET-29b vector (Novagen) at the sites of the same restriction enzymes. The amplified product comprising the sequence corresponding to amino acids 2188 to 2551 was digested by restriction enzymes BamHI and XhoI (TaKaRa) and inserted into pET28a vector (Novagen) at the sites of the same restriction enzymes.

TABLE 2

Primers for Production of Recombinant Proteins Used as Antigens

| | | |
|---|---|---|
| Primers | Sequence | Seq. No. |

| | | |
|---|---|---|
| hFex2-5 sense | 5′-caa atg tgt cga cct cca ctt cca g-3′ | 76 |
| hFex2-5 antisense | 5′-ccc gtc caa gct tgc aca gtg tcc t-3′ | 77 |
| hFex2-2 sense | 5′-ata agg atc cac cat ttt tgt tcc aa-3′ | 78 |
| hFex2-2 antisense | 5′-aat aac tcg aga ctg gga atg aga ac-3′ | 79 |

The expression vectors produced as described above were designated as ‘pET-FEX2-5’ and ‘pET-FEX2-2’, and used for the transformation of E. coli BL21 DE3. The transformed E. coli was cultured in an LB medium comprising 50 mg/ml of kanamycin. In order to induce the expression of recombinant proteins, 1 mM IPTG (isopropyl-D(−)-thiogalactopyranoside) was added thereto, when the absorbance at 600 nm reached 0.5-0.6. Then, E. coli was further cultured at 37° C. for 3 hours. Then, expressed proteins were purified in a conventional manner (Kim, J.-E. et al., J. Cell. Biochem., 77:169-187, 2000); First, the culture of the transformed, E. coli was subjected to centrifugal separation to obtain cells, and the cells were resuspended in a lysis buffer solution (50 mM Tris-HCl (pH 8.0), 100 mM NaCl, 1 mM EDTA, 1% Triton X-100, 1 mM PMSF, 0.5 mM DTT). The cell suspension was homogenized with ultrasonic waves, proteins expressed in the form of inclusion bodies were dissolved in 8M uric acid-modified buffer, and then the modified proteins were purified by using a Ni-NTA resin (Qiagen). The recombinant proteins were eluted with 200 mM imidazole solution, and then purified in 20 mM Tris-HCl buffer comprising 50 mM sodium chloride, by way of the dialysis using urea with a concentration varying from a high concentration to a low concentration. The purified recombinant proteins were determined by SDS-PAGE (The results are not shown).

Two kinds of recombinant proteins (200 μg/ml) produced as described above were injected to different rabbits to raise antibodies. More particularly, 1 ml of each recombinant protein was mixed with the same amount of the complete Freund's adjuvant and injected to a rabbit. Then, each recombinant protein was mixed with the same amount of the incomplete Freund's adjuvant and injected to a rabbit through a subcutaneous route, once in two weeks for the total period of 8 weeks. After antibodies were raised, blood was obtained from the rabbits. After the blood collection, the blood sample was left at room temperature for about 2 hours and subjected to centrifugal separation at 1000×g under a at 4° C. for 30 minutes, in order to isolate anti-sera. The anti-sera were purified with Protein A Sepharose (Amersham Pharmacia) according to the manufacturer's protocol to obtain immunoglobulin fractions.

<2-2> Production of Monoclonal Antibody Against Human FEX-2

To produce a monoclonal antibody against human FEX-2, cDNA comprising a sequence corresponding to amino acids 1173 to 1727 from the complete human FEX-2 amino acid sequence (sequence No. 1) was produced. The cDNA was obtained by means of PCR using the pcDNA-Fex2 DNA according to Example <1-1>, as a template, and the primers (sequence Nos. 80 and 81) as described hereinafter (Table 3). The PCR reaction was performed under the following condition: 2 min at 95° C.; and 25 cycles of, 30 sec at 94° C., 30 sec at 60° C. and 30 sec at 72° C. The amplified product was digested by restriction enzymes BamHI and XhoI (TaKaRa) and inserted into pET43.1a vector (Novagen) at the sites of the same restriction enzymes.

TABLE 3

Primers for Production of Recombinant Proteins Used as Antigens

| | | |
|---|---|---|
| Primers | Sequence | Sequence. No. |

| | | |
|---|---|---|
| HFex2-4E5 sense | 5′-aaa aag gat cca cag tgt ttg ctc c-3′ | 80 |
| HFex2-4E5 antisense | 5′-ttt tac tcg aga ctt ttg gga gat agc-3′ | 81 |

The expression vector constructed as described above was designated as ‘pET-4E5’ and subjected to the same procedures of protein expression and isolation as described in the above Example <2-1>. All procedures for the monoclonal antibody production were performed by Dinona Inc. (Seoul, Korea). More particularly, six mice were immunized with 20 ug of the recombinant protein produced as described above at 2-week intervals to obtain a positive hybridoma clone (clone 5G3). The positive hybrodoma clone was injected to a mouse through an intraperitoneal route, and monoclonal antibody was obtained from the ascites. The hybridoma 5G3 was deposited in one of the international depository authorities, i.e. the Korean Collection for Type Cultures (KCTC) located within the biological resources center of the Korea Research Institute of Bioscience and Biotechnology as the Accession No. KCTC-10639BP on May 21, 2004.

The isotype of the monoclonal antibody was determined by IsoStrip mouse monoclonal antibody isotyping kit (Roche). The isotype of the inventive monoclonal antibody was shown as IgG1 with lambda chain.

<2-3> Determination of Expression of FEX-2 Protein Using Human FEX-2 Monoclonal Antibody in Human Spleen Tissue and L/FEX-2 Cells

The monoclonal antibody against the human FEX-2, produced in the above Example <2-2>, was determined by western blotting whether it can detect FEX-2 protein expressed in the human spleen tissue. Further, the Western blotting was performed to determine whether the human FEX-2 protein is expressed in the L/FEX-2 cells of the above Example <1-2>. As a control, L/Mock cells were used.

First, 5 ml of a cell lysis buffer was added to 0.5 mg of the human spleen tissue. Then, the mixture was homogenized with a tissue homogenizer and left on the ice for 1 hour to obtain a cell lysate. L/FEX-2 cells and L/Mock cells, as a control, were washed with PBS many times and a cell lysis buffer was added thereto to perform lysis of the cells. Next, the cell lysate was subjected to centrifugal separation at 4° C. under 12,000 rpm for 10 minutes to obtain the supernatant comprising lysable proteins. To determine the protein concentration, Bradford assay (BioRad, Hercules, Calif.) was performed by, using BSA as a standard. Then, 30 μg of the protein samples extracted from the spleen tissue, L/FEX-2 cells and L/Mock cells were subjected to electrophoresis on polyacrylamide gel comprising 6% SDS. The protein on the gel was transferred to a nitrocellulose membrane by using electrophoresis. The protein transferred to the membrane was subjected to a reaction with a 5% skim milk solution for 1 hour to interrupt non-specific protein conjugation. Further, monoclonal anti FEX-2 antibody (5G3) was diluted with a TBS-T (50 mM Tris-HCl, pH 7.6, 150 mM NaCl, 0.1% Tween 20) solution, and was allowed to be conjugated with a nicrocellulose membrane for at least 16 hours in a refrigerated state. After the completion of the reaction, the membrane was washed with a TBS-T solution three times. Then, an HRP-conjugated secondary antibody (HRP-conjugated-anti-mouse IgG; Santa Cruz Co., CA, USA) was further added thereto to perform conjugation at room temperature for 1 hour. After washing the membrane three times, 1 ml of ECL (chemical luminescence material) was added to the membrane to visualize the site, where an antigen-antibody reaction occurred, and the site was exposed to X-ray.

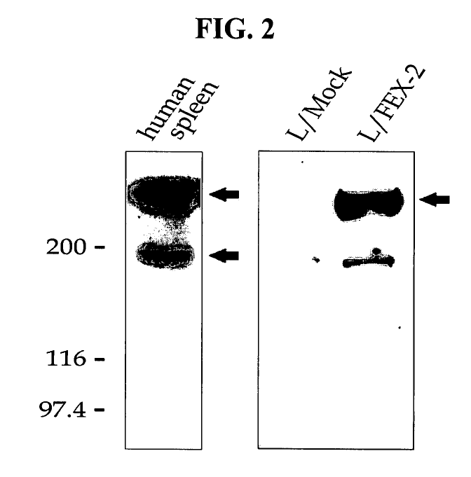

As a result of the test, bands each having a size of about 270 kDa and about 200 kDa were detected in the human spleen cells. Bands with the same sizes as described above were detected in L/FEX-2 (FIG. 2). It could be seen from the above results that the monoclonal antibody against the human FEX-2 reacts specifically with FEX-2 mL/FEX-2 cells and the spleen tissue.

### Example 3

### Determination of FEX-2 Expression on L/FEX-2 Cell Surface

<3-1> Fluorescence-Activated Cell Sorting (FACS) Analysis

The monoclonal human antibody (5G3) specific to FEX-2, produced in the above Example <2-3>, was used in FACS analysis, in order to determine whether FEX-2 was expressed on the surface of L/FEX-2 cells according to the above Example <1-2>.

A confluent plate, in which L/FEX-2 cells were cultured, was treated with PBS comprising 0.25% trypsin and 0.05% EDTA to detach the cells from the plate surface. The cells were washed with PBS twice, and resuspended in PBS. The monoclonal anti-FEX-2 antibody 5G3) was added to the cell suspension and cultured at 4° C. for 1 hour. Then, 10 μg/ml of FITC-conjugated rabbit anti-mouse secondary IgG antibody (Santa Cruz Biotechnology, Inc., CA) was added to the cell culture. Then, the cells were further cultured at 4° C. for 1 hour, and analyzed at 488 nm by using a flow cytometer equipped with a 5 watt laser (FACS Calibur system, Becton Dickinson, San Jose, Calif.). As a control, mouse immunoglobulin was used instead of the monoclonal anti-FEX-2 antibody.

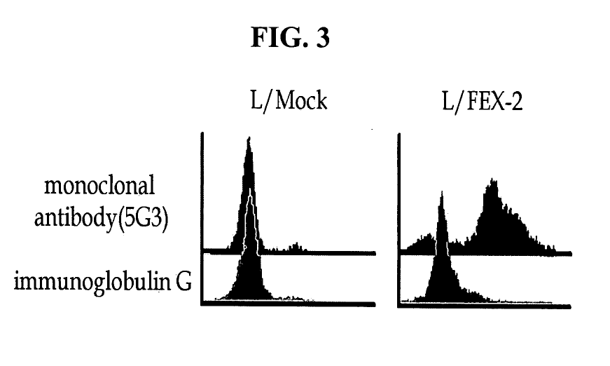

After the test, it could be seen that FEX-2 was expressed on the surface of L/FEX-2 cells. On the contrary, FEX-2 could not be expressed on the surface of L/Mock cells (FIG. 3).

<3-2> Surface Biotinylation Assay

Surface biotinylation assay was performed to determine whether FEX-2 was expressed on the surface of L/FEX-2 cells. The L/FEX-2 cells according to Example <1-2> were washed with ice-cooled phosphate buffer solution (pH 8.0) three times. Next, the cells were suspended in phosphate buffer solution to a concentration of 2.5×10⁷ cells/ml, 1 mg of sulfo-NHS-LS-biotin (Pierce) was added thereto to perform a reaction at room temperature for 30 minutes. Sulfo-NHS-Ls-biotin was not added to the control. To the reaction mixture, a immunoprecipitation buffer (which comprises 50 mM Tris-HCl, 150 mM NaCl, 1% Triton X-100, 1 mM CaCl₂, 1 mM MgCl₂ and a protease inhibitor mix (Roche); pH 7.4) was added to perform lysis of the cells. Then, the cell lysate was subjected to centrifugal separation at 4° C. under 12,000 rpm for 10 minutes to obtain the supernatant comprising lysable proteins. To reduce a non-specific reaction with beads, the cell lysate was applied to protein G beads, and allowed to react at 4° C. for 2 hours, followed by removal of the beads. Then, the resultant product was allowed to react with a protein G sepharose matrix conjugated with the polyclonal FEX-2 antibody according to the above Example <2-1> at 4° C. overnight to perform immunoprecipitation. After the beads were washed three times, 20 μl of a sample buffer was added thereto, followed by boiling. Then, the sample was subjected to electrophoresis on polyacrylamide gel comprising 6% SDS. The protein on the gel was transferred to a nitrocellulose membrane by using electrophoresis. The protein transferred to the membrane was subjected to a reaction with a 5% skim milk solution for 1 hour to interrupt non-specific protein conjugation. Further, anti FEX-2 antibody was diluted with a TBS-T solution (50 mM Tris-HCl, pH 7.6, 150 mM NaCl, 0.1% Tween 20), and was allowed to be conjugated with a nicrocellulose membrane for at least 16 hours in a refrigerated state. After the completion of the reaction, the membrane was washed with a TBS-T solution three times. Then, an HRP-conjugated secondary antibody (HRP-conjugated-anti-rabbit IgG; SantaCruz Co., CA, USA) was further added thereto to perform conjugation at room temperature for 1 hour. After washing the membrane three times, 1 ml of ECL (chemical luminescence material) was added to the membrane to visualize the site, where an antigen-antibody reaction occurred, and the site was exposed to X-ray. Additionally, the nitrocellulose membrane was conjugated with HRP-conjugated streptavidin, instead of the anti FEX-2 antibody, at room temperature for 1 hour. Then, 1 ml of ECL (chemical luminescence material) was added to the membrane to visualize the site, where an antigen-antibody reaction occurred, and the site was exposed to X-ray.

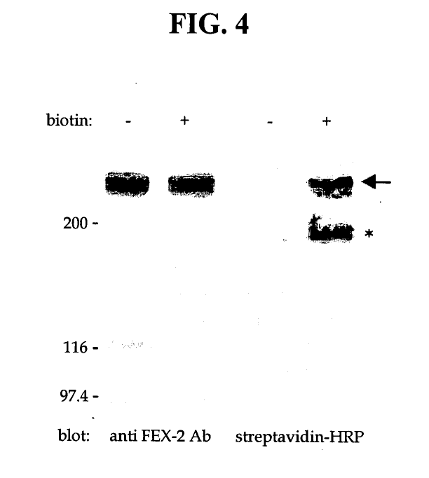

After the test, the FEX-2 protein immunoprecipitated with the FEX-2 antibody was not detected by streptavidin but by the FEX-2 antibody, in the control that was not treated with biotin. On the contrary, the FEX-2 protein was detected by streptavidin as well as the anti FEX-2 antibody, in the group treated with biotin (FIG. 4).

It could be seen from the above results that FEX-2 protein present on the surface of cells is biotinylated through the treatment with biotin, and then the biotinylated product was immunoprecipitated by the FEX-2 antibody-conjugated beads and could be detected by the anti FEX-2 antibody and streptavidin. On the contrary, in the case of the control that was not treated with biotin, FEX-2 protein present on the surface of cells could not be detected by streptavidin due to the absence of biotinylation of the FEX-2 protein. Therefore, it could be seen that FEX-2 protein was present on the surface of L/FEX-2 cells.

### Example 4

### Identification of Tissue Capable of Expression of FEX-2 by Immunostaining

To examine how FEX-2 was expressed, immunostaining assays were performed for about 20 kinds of tissues (obtained from the Pathology room of the Kyung-pook National Univ.) including the human liver, spleen, pancreas, heart, testis, lung, lymph node, ovary, skin and adrenal gland by using the polyclonal human FEX-2 antibody obtained from the above Example <2-1>.

First, each tissue was dipped in 3.7% paraformaldehyde, fixed overnight at room temperature, and then cut 5 μm intervals. Next, such treated tissues were dipped in xylene and alcohol individually for about 5 minutes, and treated with 0.5% hydrogen peroxide for 10 minutes in order to inhibit the intrinsic peroxydase activity. To interrupt a non-specific reaction, each tissue fragment was left in 50 mM NH₄Cl for 30 minutes, a blocking solution (1×PBS, 1% BSA, 0.05% saponin and 0.2% gelatin) was added thereto, and the resultant mixture was allowed to react at 4° C. for 1 hour. Then, such treated tissues were allowed to be conjugated with the polyclonal human FEX-2 antibody overnight at 4° C. After the reaction, the tissues were washed three times, and a chemical luminescence agent was added thereto to visualize the site, where an antigen-antibody reaction occurred. As a control, the antibody pre-cultured with the antigen was used.

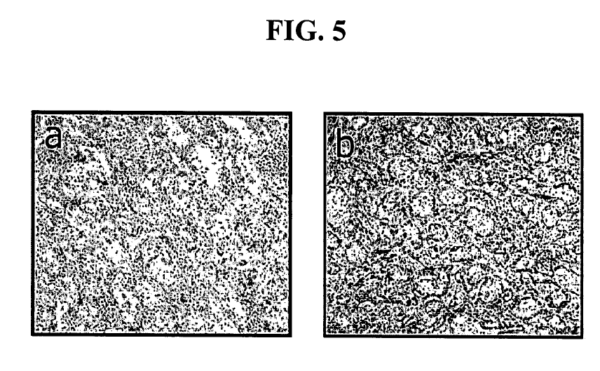

After the test, it could be seen that FEX-2 protein was expressed in the venous sinus of the human spleen (see FIG. 5). Also, FEX-2 was expressed in the maxillary sinus of lymph node and sinusoidal blood vessel of the liver (not shown). According to the above result, it could be estimated that FEX-2 was expressed in sinusoidal endothelial cells to interact with cells present in the blood.

### Example 5

### Assay for Lymphocyte Adhesion to FEX-2

To examine the activity of lymphocyte adhesion to FEX-2, peripheral blood lymphocytes were isolated from the blood by using Ficoll (Pharmacia Biotech) gradient centrifugation (Johenson-Leger C. A. et al., Blood, 100, 2479-2486, 2002). After the L/FEX-2 cells obtained from Example <1-2> and L/Mock cells as a control were cultured confluently in a 12-well plates comprising a DMEM medium, 1×10⁵ lymphocytes labeled with DiI fluorescence dye (i.e. molecular probe) were added thereto and the cells were cultured at 37° C. for 30 minutes. Then, the cells were washed with the same medium three times, and observed with an optical microscope, at 10 randomly selected positions, under HMMF (high magnification fields, ×400) to count the number of lymphocytes adhered to the L/FEX-2 cells.

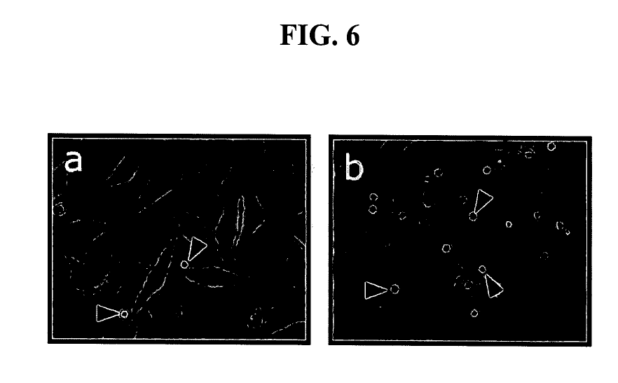

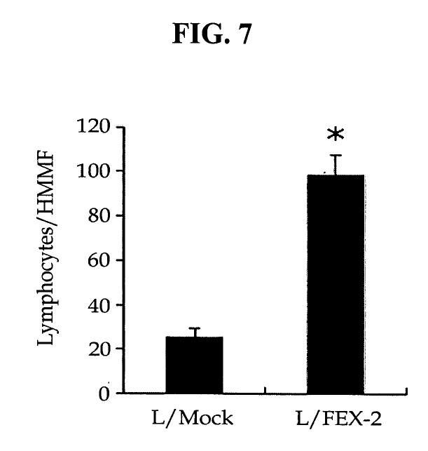

After the test, it could be seen that a greater number of lymphocytes were adhered to the L/FEX-2 cells that expresses FEX-2, when compared to L/Mock cells as a control (FIGS. 6 and 7).

### Example 6

### Inhibition Against Lymphocyte Adhesion to FEX-2, Induced by Monoclonal FEX-2 Antibody

To determine whether FEX-2 mediated lymphocyte adhesion to the L/FEX-2 cells, an antibody specific to FEX-2 was examined for inhibition against lymphocyte adhesion to the L/FEX-2 cells.

First, the L/FEX-2 cells, according to the above Example 1 were cultured in a 35 mm plate, and added to the monoclonal FEX-2 antibody (5G3) according to the above Example <2-2>, followed by pre-culture at 37° C. for 30 minutes. Next, lymphocytes labeled with DiI fluorescence dye (Molecular Probe) was added to the pre-cultured cells, followed by culture at 37° C. for 30 minutes. Then, lymphocyte adhesion to L/FEX-2 was examined according to the same manner as described in the above Example 5. As a control, immunoglobulin G was used instead of the monoclonal FEX-2 antibody.

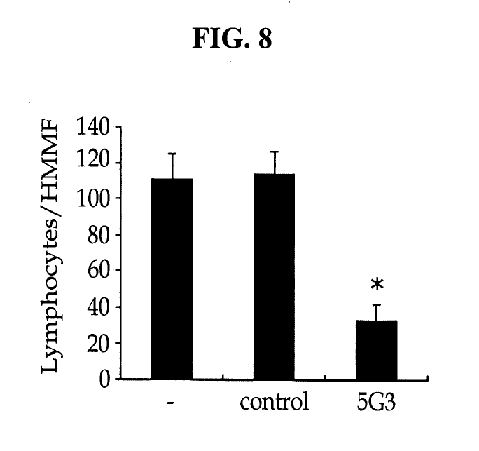

As a result, it could be seen that lymphocyte adhesion to the surface of L/FEX-2 cells could be inhibited specifically by the monoclonal FEX-2 antibody (FIG. 8)

### Example 7

### Identification of Integrin Receptors

<7-1> Divalent Cation Dependence of Lymphocyte Adhesion to FEX-2

Integrin receptors that mediate lymphocyte adhesion to cells require divalent cations. Based on this fact, divalent cation (Mn²⁺, Mg²⁺ and ca²⁺) dependence of lymphocyte adhesion to FEX-2 was examined. To perform the examination, CaCl₂, MgCl₂ and MnCl₂ were added to calcium- and magnesium-free HBSS (Hank's Balanced Salt Solution), each in an amount of 2 mM. The divalent cation-containing solution was added to the culture of Lymphocytes and L/FEX-2 according to Example 5. After culturing, the number of lymphocytes adhered to the L/FEX-2 cells was counted in the same manner as described in Example 5.

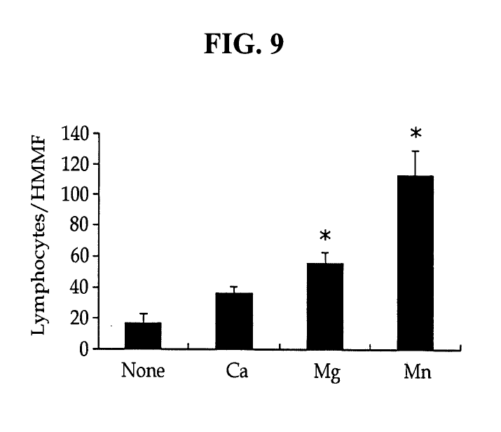

After the test, it could be seen that addition of Mn²⁺ enhanced lymphocyte adhesion to L/FEX-2 at the highest degree and addition of Mg²⁺ provided the second highest degree of enhancement. Although, Ca²⁺ enhances lymphocyte adhesion activity, there was no significant difference as compared to the control (FIG. 9).

<7-2> Identification of Integrin Receptors Participating in Lymphocyte Adhesion to FEX-2

To identify integrin receptors participating in lymphocyte adhesion to FEX-2, various types of antibodies were used to perform cell adhesion inhibition assay.

Various types of monoclonal integrin-specific antibodies (Chemicon, International Inc., Temecula, Calif.) (individually 10 μg/ml) were pre-cultured with lymphocytes (3×10⁵ cells/ml) in 1 ml of culture solution at 37° C. for 30 minutes. The antibodies used in this Example were as follows: P5D2 (antibody against 01), 25.3 (antibody against αL), Bearl (antibody against αM) and 3.9 (antibody against αX). As a control, pre-culture with normal mouse immunoglobulin was used instead of the monoclonal integrin-specific antibody. Then, the lymphocytes pre-cultured with each antibody were added to the cultured L/FEX-2 cells, followed by culture at 37° C. for 30 minutes. The cells adhered thereby were determined in the same manner as described in Example 5.

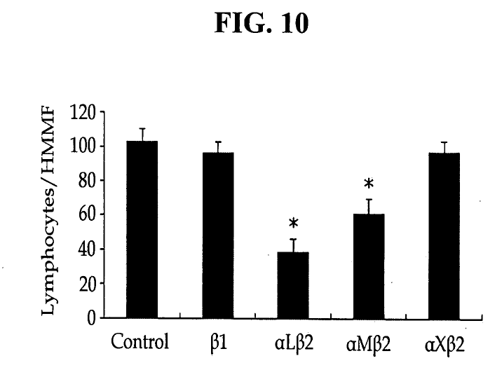

After the test, lymphocyte adhesion to FEX-2 is inhibited specifically by antibodies against αL and αM integrin, while not inhibited by antibodies against αX and β1 integrin (FIG. 10).

Therefore, it could be seen that lymphocyte adhesion to the cells with FEX-2 protein was mediated by αLβ2 and αMβ2 integrin.

### Example 8

### Inhibition of Lymphocyte Adhesion to FEX-2, Induced by Recombinant Deletion Mutant FEX-2 Protein

<8-1> Inhibition of Lymphocyte Adhesion to FEX-2, Induced by Four FEX-2 Subunits

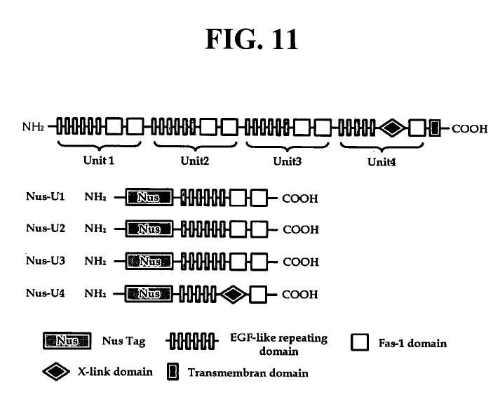

The extracellular part of FEX-2 was divided into four subunits, Nus-U1, Nus-U2, Nus-U3 and Nus-U4 (FIG. 11). Herein, Nus-U1, Nus-U2 and Nus-U3 have a very similar domain structure. More particularly, each of Nus-U1, Nus-U2 and Nus-U3 has one EGF-like repeating domain and two fas-1 domains. Nus-U4 has one EGF-like repeating domain, one fas-1 domain and one X-link domain.

To produce recombinant proteins Nus-U1, Nus-U2, Nus-U3 and Nus-U4, each of cDNA fragments encoding amino acids 66-655, 691-1268, 1303-1883 and 1913-2449 was generated by using PCR amplification using pcDNA-fex2 DNA as a template and the following primers (sequence Nos. 82-89) (Table 4). The PCR reaction was performed under the following conditions: 2 min at 95° C.; and 25 cycles of, 30 sec at 94° C., 30 sec at 60° C. and 30 sec at 72° C. Then, each amplified product was digested by restriction enzymes BamHI (TaKaRa) and XhoI (TaKaRa), and then inserted into pET-43.1a vector (Novagen) at the sites of the same restriction enzymes. The expression vectors were designated as ‘pET-U1’, ‘pET-U2’, ‘pET-U3’ and ‘pET-U4’. Then, E. coli was transformed with the expression vectors in the same manner as described in Example <2-1> to induce expression of proteins. The proteins were isolated and purified to provide proteins Nus-U1, Nus-U2, Nus-U3 and Nus-U4.

TABLE 4

Primers for Production of cDNAs of Nus-U1, Nus-U2, Nus-U3 and Nus-U4

| | | |
|---|---|---|
| Primers | Sequence | Seq. No. |

| | | |
|---|---|---|
| Nus-U1 sense | 5′-aaa aag gat ccg tag ggg ttc gag att g-3′ | 82 |
| Nus-U1 antisense | 5′-aat aac tcg aga ctg gga gga atg aga ac-3′ | 83 |
| Nus-U2 sense | 5′-aaa aag gat cca act ctg agc cca cag-3′ | 84 |
| Nus-U2 antisense | 5′-aaa tac tcg agt gac tga att tcc aga act ttt ccc-3′ | 85 |
| Nus-U3 sense | 5′-aaa aag gat ccg aga aga gga gat gc-3′ | 86 |
| Nus-U3 antisense | 5′-ttt tac tcg aga cta tca atc agc aga c-3′ | 87 |
| Nus-U4 sense | 5′-aaa atg gat cca ggt gga gta aac caa ag-3′ | 88 |
| Nus-U4 antisense | 5′-aaa tag aat tct cag ggt gct ttt aaa ggc-3′ | 89 |

To determine whether the above recombinant proteins Nus-U1, Nus-U2, Nus-U3 and Nus-U4 inhibit lymphocyte adhesion, 10 μM of each of the recombinant proteins Nus-U1, Nus-U2, Nus-U3 and Nus-U4 was added to lymphocytes labeled with DiI fluorescence dye to perform pre-culture at 37° C. for 30 minutes. Then, the pre-cultured lymphocytes were added to the L/FEX-2 cells expressing FEX-2, cultured at 37° C. for 30 minutes, and was examined for lymphocyte adhesion to L/FEX-2 in the same manner as described in Example 5. As a control, Nus protein was used.

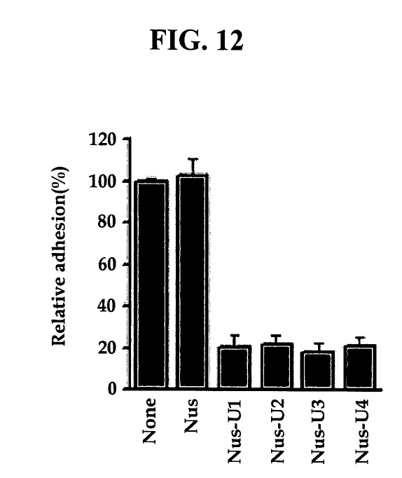

As a result, it could be seen that pre-culture of lymphocytes with recombinant proteins Nus-U1, Nus-U2, Nus-U3 and Nus-U4 significantly inhibited lymphocyte adhesion to FEX-2 (FIG. 12).

<8-2> Inhibition of Lymphocyte Adhesion to FEX-2, Induced by Recombinant Proteins Nus-EGF3, Nus-Fas5 and Nus-Fas6

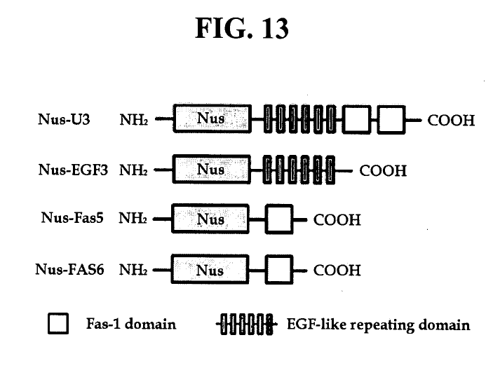

Nus-U3, one of the subunits described in Example <8-1> was further divided into Nus-EGF3, Nus-Fas5 and Nus-Fas6, and the recombinant proteins were examined for lymphocyte adhesion to FEX-2. Nus-EGF3 was a polypeptide comprising the third EGF-like repeating domain in the FEX-2 protein. Nus-Fas5 and Nus-Fas6 were polypeptides comprising the fifth and sixth fas-1 domains, respectively (FIG. 13).

To produce Nus-EGF3, Nus-Fas5 and Nus-Fas, which were deletion mutant proteins of the FEX-2 protein, each of cDNA fragments encoding amino acids 1301-1596, 1631-1727, and 1778-1883 was generated by using PCR amplification using pcDNA-FEX2 described in Example 1 as a template and the following primers (sequence Nos. 90-95) (Table 5). The PCR reaction was performed under the following conditions: 2 min at 95° C.; and 25 cycles of, 30 sec at 94° C., 30 sec at 60° C. and 30 sec at 72° C. Then, each amplified product was digested by restriction enzymes BamHI (TaKaRa) and XhoI (TaKaRa), and then inserted into pET-43.1a vector (Novagen) at the sites of the same restriction enzymes. The expression vectors were designated as ‘pET-EGF3’, ‘pET-Fas5’ and ‘pET-Fas6’. Then, E. coli was transformed with the expression vectors in the same manner as described in Example <2-1> to induce expression of proteins. The proteins were isolated and purified to provide proteins Nus-EGF3, Nus-Fas5 and Nus-Fas6.

TABLE 5

Primers for Production of cDNAs of Nus-EGF3, Nus-Fas5 and Nus-Fas6

| | | |
|---|---|---|
| Primers | Sequence | Seq. No. |

| | | |
|---|---|---|
| Nus-EGF3 sense | 5′-aaa aag gat ccg aga aga gga gat gc-3′ | 90 |
| Nus-EGF3 antisense | 5′-aaa aac tcg agt cag gat tta cct gcc gc-3′ | 91 |
| Nus-Fas5 sense | 5′-ttt tag gat cca ctg ttt ttg cac c-3′ | 92 |
| Nus-Fas5 antisense | 5′-ttt tac tcg aga ctt ttg gga gat agc-3′ | 93 |
| Nus-Fas6 sense | 5′-ttt tag gat cca ctc tct tct ggc c-3′ | 94 |
| Nus-Fas6 antisense | 5′-ttt tac tcg aga cta tca atc agc aga c-3′ | 95 |

To determine whether the above recombinant proteins Nus-EGF3, Nus-Fas5 and Nus-Fas6 inhibit lymphocyte adhesion, 10 μM of each of the recombinant proteins of Nus-EGF3, Nus-Fas5 and Nus-Fas6 was added to lymphocytes labeled with DiI fluorescence dye to perform pre-culture at 37° C. for 30 minutes. Then, the pre-cultured lymphocytes were added to the L/FEX-2 cells expressing FEX-2, cultured at 37° C. for 30 minutes, and was examined for lymphocyte adhesion to L/FEX-2 in the same manner as described in Example 5. As a control, Nus protein was used.

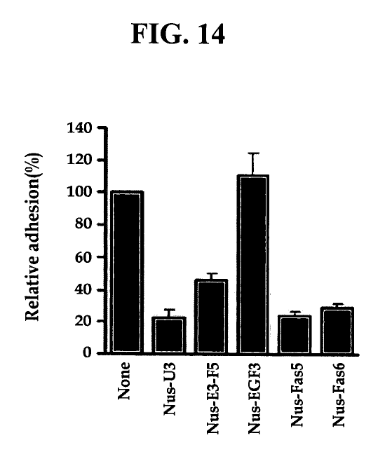

As a result, it could be seen that addition of polypeptides comprising fas-1 domains, i.e. Nus-Fas5 and Nus-Fas6 significantly inhibited lymphocyte adhesion to FEX-2. However, addition of a recombinant protein comprising an EGF-like domain had little effect upon lymphocyte adhesion FIG. 14).

### Example 9

### Inhibition of Lymphocyte Adhesion Depending on Concentration of fas-1 Domain-Comprising Polypeptide

In the same manner as described in Example 8, Nus-U3, Nus-EGF3 and Nus-Fas5 were examined for degrees of inhibition against lymphocyte adhesion, wherein the concentration of each protein was varied to 0.1, 1 and 10 μM.

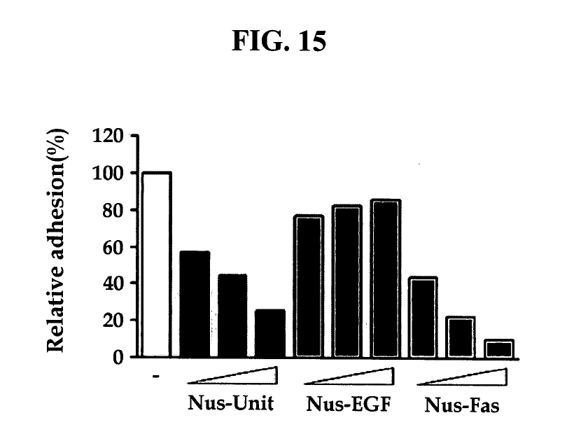

As a result, as the concentration of a recombinant protein comprising fas-1 domain increased, lymphocyte adhesion also increased. On the contrary, as the concentration of a recombinant protein comprising an EGF-like domain increased, there was no significant effect upon lymphocyte adhesion except a slight increase in lymphocyte adhesion (FIG. 15).

### Example 10

### Inhibition of Lymphocyte Adhesion to FEX-2, Induced by Various Polypeptides Comprising fas-1 Domains

<10-1> Inhibition of Lymphocyte Adhesion to FEX-2, Induced by fas-1 Domains in FEX-2 Protein

All fas-1 domains presented in the FEX-2 protein were examined for inhibition against lymphocyte adhesion to FEX-2.

To produce recombinant proteins from all fas-1 domains comprised in the FEX-2 protein, each of cDNA fragments encoding amino acids 406-508, 554-655, 1030-1130, 1173-1268 and 2356-1449 was generated by using PCR amplification using pcDNA-Fex2 DNA, as a template, and the following primers (sequence Nos. 96-105) (Table 6). The PCR reaction was performed under the following conditions: 2 min at 95° C.; and 25 cycles of, 30 sec at 94° C., 30 sec at 60° C. and 30 sec at 72° C. Then, each fas-1 domain of the amplified FEX-2 protein, except the seventh fas-1 domain, was digested by restriction enzymes BamHI (TaKaRa) and XhoI (TaKaRa), and then inserted into pET-43.1a vector (Novagen) at the sites of the same restriction enzymes. The seventh fas-1 domain of the amplified FEX-2 protein was digested by restriction enzymes BamHI (TaKaRa) and EcoRI (TaKaRa), and then inserted into pET-43.1a vector (Novagen) at the sites of the same restriction enzymes. The expression vectors were designated as ‘pET-Fas1’, ‘pET-Fas2, ‘pET-Fas3’, ‘pET-Fas4’ and ‘pET-Fas7’. Then, E. coli was transformed with the expression vectors in the same manner as described in Example <2-1> to induce expression of proteins. The proteins were isolated and purified to provide proteins Nus-Fas1, Nus-Fas2, Nus-Fas3, Nus-Fas4 and Nus-Fas7.

TABLE 6

Primers for Production of cDNAs of fas-1 domains Comprisied in FEX-2 Protein

| | | |
|---|---|---|
| Primers | Sequence | Seq. No. |

| | | |
|---|---|---|
| Nus-Fas1 sense | 5′-aaa aag gat cca cgg tgc tgt tac c-3′ | 96 |
| Nus-Fas1 antisense | 5′-aaa aac tcg aga ctt aac ttg tcc atg gct c-3′ | 97 |
| Nus-Fas2 sense | 5′-ata agg atc cac cat ttt tgt tcc aa-3′ | 98 |
| Nus-Fas2 antisense | 5′-aat aac tcg aga ctg gga gga atg aga ac-3′ | 99 |
| Nus-Fas3 sense | 5′-ttt acg gat cca ctg tcc tcg tgc ctt c-3′ | 100 |
| Nus-Fas3 antisense | 5′-ttt aac tcg aga ctt tgt ggg acc agc-3′ | 101 |
| Nus-Fas4 sense | 5′-aaa aag gat cca cag tgt ttg ctc c-3′ | 102 |
| Nus-Fas4 antisense | 5′-aaa tac tcg agt gac tga att tcc aga act ttt ccc-3′ | 103 |
| Nus-Fas7 sense | 5′-ttt aag gat cca ccc tct ttg tgc cac-3′ | 104 |
| Nus-Fas7 antisense | 5′-aaa tag aat tct cag ggt gct ttt aaa ggc-3′ | 105 |

Then, Example <8-2> was repeated to determine whether Nus-Fas1, Nus-Fas2, Nus-Fas3, Nus-Fas4 and Nus-Fas7, produced in this Example, as well as Nus-Fas5 and Nus-Fas6, produced in the above Example <8-2>, inhibited lymphocyte adhesion to FEX-2.

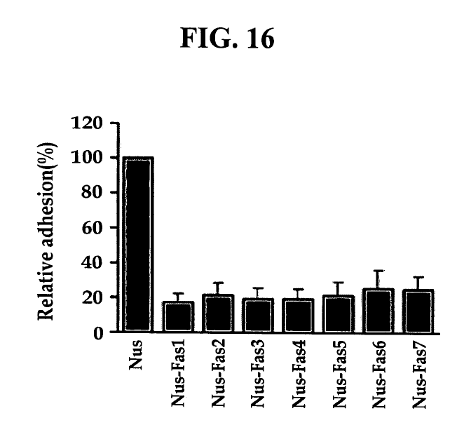

As a result, it could be seen that recombinant proteins comprising all fas-1 domains present in FEX-2 inhibited lymphocyte adhesion (FIG. 16).

<10-2> Inhibition Against Lymphocyte Adhesion to FEX-2 of fas-1 Domain Contained in M. Tuberculosis Protein

In this Example, fas-1 domains present in M. Tuberculosis proteins mpt83 and mpt70 were examined for inhibition against lymphocyte adhesion to FEX-2. First, cDNA fragment encoding amino acids 130-256 of mpt 70 protein and that encoding amino acids 123-218 of mpt83 protein were generated by using RT-PCR using RNA of M. Tuberculosis as a template and the following primers (sequence Nos. 106-109) (Table 7). Herein, extraction of RNA from M. Tuberculosis was performed using trizol (Invitrogen) according to the manufacturer's instructions. The PCR reaction was performed under the following conditions: 2 min at 95° C.; and 25 cycles of, 30 sec at 94° C., 30 sec at 60° C. and 30 sec at 72° C. Then, each fas-1 domain of the amplified mpt70 and mpt83 proteins was digested by restriction enzymes BamHI (TaKaRa) and XhoI (TaKaRa), and then inserted into pET-28a vector (Novagen) at the sites of the same restriction enzymes. The expression vectors were designated as ‘pET-mpt70’ and ‘pET-mpt83’. Then, E. coli was transformed with the expression vectors in the same manner as described in Example <2-1> to induce expression of proteins. The proteins were isolated and purified to provide proteins Nus-mpt70 and Nus-mpt83.

TABLE 7

Primers for Production of cDNAs of fas-1 domains Comprisied in
M. Tuberculosis Proteins

| | | |
|---|---|---|
| Primers | Sequence | Seq. No. |

| | | |
|---|---|---|
| Nus-mpt70 sense | 5′-aaa aag gat cca cgg tgt tcg cac-3′ | 106 |
| Nus-mpt70 antisense | 5′-aaa atc tcg agt cac gga ggc att agc-3′ | 107 |
| Nus-mpt83 sense | 5′-aat tag gat cca ccg ttt tcg ccc-3′ | 108 |
| Nus-mpt83 antisense | 5′-aaa atc tcg agt cac ggg ggc atc ag-3′ | 109 |

In the same manner as described in Example <8-2>, Nus-mpt70 and Nus-mpt83 were examined for inhibition against lymphocyte adhesion to FEX-2, wherein each of Nus-mpt70 and Nus-mpt83 protein was added at a concentration of 0.1, 1 and 10 μM.

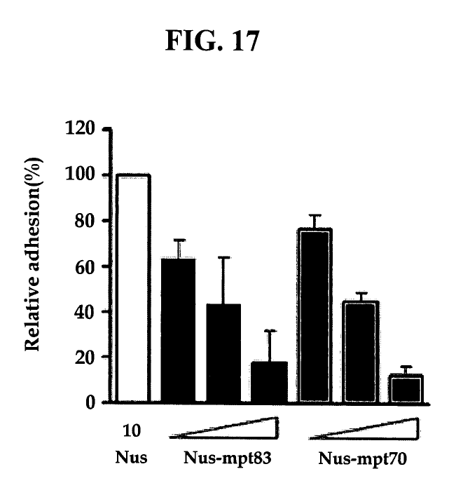

As a result, it could be seen that polypeptides comprising fas-1 domains in M. Tuberculosis proteins mpt83 and mpt70 inhibited lymphocyte adhesion to FEX-2. Additionally, as the concentration of the polypeptide increased, lymphocyte adhesion also increased (FIG. 17).

<Application 1> Arthritis

Arthritis is caused by an autoimmune abnormality, and results in breakdown of cartilage due to chronic inflammation generated in the synovia cavity of joints during its progress. Arthritis includes infective arthritis, degenerative arthritis, rheumatoid arthritis, femoral head avascular necrosis, ankylosing spondylitis, arthritis caused by congenital malformation, or the like. It is known that all types of arthritis arise chronic inflammation in the synovia cavity while the disease progresses. Also, it is reported that an inflammation reaction occurs primarily or secondarily, and thus causes breakdown of cartilage, thereby significantly affecting the progress of a disease. Herein, introduction of lymphocytes into a joint through the interaction with endothelial cells functions as an important pathological mechanism (Haskard D. O. Curr. Opin. Rheumatol. 7:229-34, 1995). When treating arthritis, inhibition of pains and inflammation conditions, resulting in a decrease of the breakdown rate of joints or muscles and minimization of functional loss, takes precedence over the causative therapy. Therefore, the pharmaceutical composition according to the present invention is very efficient for prevention and treatment of arthritis.

<Application 2> Diabetic Ophthalmic Disease

Diabetic ophthalmic diseases are one of the main complications of diabetes and may result in blindness. Diabetic ophthalmic diseases could occur, when diabetes continues for a long time regardless of blood sugar control. Recently, as therapy for diabetes is improved, patients suffering from diabetes will have an extended lifetime, followed by an increase in prevalence of diabetic retinopathy increases. Therefore, diabetic retinopathy is the most serious cause of the adult blindness in Korea as well as Europe. It is reported that patients suffering from diabetic retinopathy show increased amount of cell adhesion molecules, and such cell adhesion molecules cause leukostasis, non-perfusion, vascular leakage and endothelial cell damage (Miyamoto K. Proc. Natl. Acad. Sci. USA. 96:10836-41, 1999; Joussen A. M. Am. J. Pathol. 158:147-52, 2001; Barouch F. C. Invest Opthalmol. Vis. Sci. 41(5):1153-8, 2000). Particularly, it was reported that inflammation reactions induced by lymphocyte adhesion plays an important role in diabetic ophthalmic disease such as diabetic retinopathy (Joussen A. M. FASEB J. 18:1450-1452, 2004). Therefore, the pharmaceutical composition according to the present invention is very efficient for prevention and treatment of diabetic ophthalmic disease.

<Application 3> Inflammation

Inflammation is a response of a living tissue with blood vessels against a local damage. Although an inflammatory disease may result from various causes such as an infection and wound, inflammatory diseases show similar variations regardless of causes and tissues responding inflammation. Such variations include increased blood flow, increased vessel wall permeability and lymphocyte infiltration. It was reported that cell adhesion molecules participate in all of the variations (Jackson, J. R. et al., FASEB, J. 11:457-465, 1997). Inflammation is a mechanism for restoration against damages, and thus is not a harmful response. However, an inadequate or excessive inflammation response, such as autoimmune, may result in a damage and deformation of tissues. The composition for inhibiting inflammatory diseases according to the present invention is efficient for controlling such inadequate or excessive inflammation responses.

### INDUSTRIAL APPLICABILITY

As can be seen from the foregoing, the method and pharmaceutical composition for preventing or treating inflammatory diseases according to the present invention, inhibit lymphocyte adhesion to a FEX-2 polypeptide and lymphocyte adhesion to endothelial cells, and thus can prevent or treat inflammatory diseases. Additionally, the screening method according to the present invention, which determines whether a test agent inhibits lymphocyte adhesion to FEX-2 polypeptide, allows screening of an inhibitor against lymphocyte adhesion to endothelial cells or screening of a treating agent for inflammatory diseases.

What is claimed is:

1. A method for inhibiting lymphocyte adhesion to an endothelial cell, which comprises administering to a subject in need thereof an inhibitor against lymphocyte adhesion to a FEX-2 polypeptide.

2. The method according to claim 1, wherein the FEX-2 polypeptide is derived from a mammal.

3. The method according to claim 1, wherein the FEX-2 polypeptide comprises an amino acid sequence represented by SEQ ID NO: 1 or SEQ ID NO: 9.

4. The method according to claim 1, wherein the inhibitor against lymphocyte adhesion to a FEX-2 polypeptide is selected from the group consisting of polypeptide comprising fas-1 domains, anti-FEX-2 antibody, triple helix forming agent, ribozyme, double-stranded RNA homolog to a FEX-2 mRNA target molecule and an antisense nucleic acid of FEX-2 genes.

5. The method according to claim 4, wherein the fas-1 domain is derived from a mammal.

6. The method according to claim 5, wherein the mammal is selected from the group consisting of human being, rat and mouse.

7. The method according to claim 4, wherein the fas-1 domain is derived from a protein selected from the group consisting of FEX-2, mpt70, mpt83, βig-h3, periostin and FEX-1.

8. The method according to claim 7, wherein the polypeptide comprising fas-1 domains has an amino acid sequence selected from the group consisting of SEQ ID NO: 1 to SEQ ID NO: 66.

9. The method according to claim 8, wherein the polypeptide comprising fas-1 domains has an amino acid sequence selected from the group consisting of SEQ ID NO: 1 to SEQ ID NO: 14.

10. The method according to claim 4, wherein the anti-FEX-2 antibody is a polyclonal or a monoclonal antibody.

11. The method according to claim 10, wherein the monoclonal antibody is produced by a hybridoma (Accession No. KCTC 10639BP).

12. A method for preventing or treating an inflammatory disease, which comprises administering to a subject in need thereof an inhibitor against lymphocyte adhesion to a FEX-2 polypeptide.

13. The method according to claim 12, wherein the FEX-2 polypeptide is derived from a mammal.

14. The method according to claim 12, wherein the FEX-2 polypeptide comprises an amino acid sequence represented by SEQ ID NO: 1 or SEQ ID NO: 9.

15. The method according to claim 12, wherein the inhibitor against lymphocyte adhesion to a FEX-2 polypeptide is selected from the group consisting of polypeptide comprising fas-1 domain, anti-FEX-2 antibody, triple helix forming agent, ribozyme, double-stranded RNA homolog to a FEX-2 mRNA target molecule and an antisense nucleic acid of FEX-2 gene.

16. The method according to claim 15, wherein the fas-1 domain is derived from a mammal.

17. The method according to claim 16, wherein the mammal is selected from the group consisting of a human being, rat and mouse.

18. The method according to claim 15, wherein the fas-1 domain is derived from a protein selected from the group consisting of FEX-2, mpt70, mpt83, βig-h3, periostin and FEX-1.

19. The method according to claim 18, wherein the polypeptide comprising fas-1 domains has an amino acid sequence selected from the group consisting of SEQ ID NO: 1 to SEQ ID NO: 66.

20. The method according to claim 19, wherein the polypeptide comprising fas-1 domains has an amino acid sequence selected from the group consisting of SEQ ID NO: 1 to SEQ ID NO: 14.

21. The method according to claim 15, wherein the anti-FEX-2 antibody is a polyclonal or a monoclonal antibody.

22. The method according to claim 21, wherein the monoclonal antibody is produced by a hybridoma (Deposit No. KCTC 10639BP).

23. The method according to claim 12, wherein the inflammatory disease is selected from the group consisting of: inflammation, inflammatory bowl disease, diabetic ocular disease, peritonitis, osteomyelitis, cellulitis, meningitis, encephalitis, pancreatitis, trauma causing shock, bronchial asthma, rhinitis, sinusitis, otitis media, pneumonia, gastritis, enteritis, cystic fibrosis, apoplexy, bronchitis, bronchiolitis, hepatitis, nephritis, arthritis, gout, spondylitis, Reiter's syndrome, polyarteritis nodosa, hypersensitivity vasculitis, Wegener's granulomatosis, polymyalgia rheumatica, giant cell arteritis, calcium crystal deposition arthropathy, pseudogout, nonarticular rheumatism, bursitis, tenosynovitis, epicondylitis (Tennis elbow), neuropathic joint disease (Charcot's joint), hemarthrosis, Henoch-Schonlein Purpura, hypertrophic osteoarthropaihy, multicentric reticulohistiocytoma, scoliosis, hemochromoatosis, sickle cell disease and other hemoglobinopathies, hyperlipoproteinemia, hypogammaglobulinemia, hyperparathyroidism, acromegaly, familial mediterranean fever, Behcet's disease, systemic lupus erythematosus, relapsing fever, psoriasis, multiple sclerosis, septicemia, septic shock, acute respiratory distress syndrome, multiple organ failure, chronic obstructive pulmonary disease, acute lung injury and broncho-pulmonary dysplasia.

24. A pharmaceutical composition for inhibiting lymphocyte adhesion to endothelial cells, which comprises an inhibitor against lymphocyte adhesion to a FEX-2 polypeptide, and pharmaceutically acceptable carrier.

25. A pharmaceutical composition for preventing or treating an inflammatory disease, which comprises an inhibitor against lymphocyte adhesion to a FEX-2 polypeptide, and pharmaceutically acceptable carrier.

26. Use of an inhibitor against lymphocyte adhesion to FEX-2 polypeptide for the preparation of a medicament for inhibiting lymphocyte adhesion to endothelial cells.

27. Use of an inhibitor against lymphocyte adhesion to a FEX-2 polypeptide for the preparation of a medicament for preventing or treating an inflammatory disease.

28. A method for screening a medicament for inhibiting lymphocyte adhesion to an endothelial cell, which comprises the steps of:
(a) pre-culturing cells expressing a FEX-2 polypeptide with or without a test agent;
(b) adding lymphocytes to the cells pre-cultured with or without a test agent in the step (a) and further culturing them; and
(c) measuring a degree of lymphocyte adhesion to the cells pre-cultured with a test agent, and comparing the measured degree with a degree of lymphocyte adhesion to the cells pre-cultured without a test agent, thereby determining whether the test agent inhibits lymphocyte adhesion.

29. A method for screening a medicament for preventing or treating an inflammatory disease, which comprises the steps of:
(a) pre-culturing cells expressing a FEX-2 polypeptide with or without a test agent;
(b) adding lymphocytes to the cells pre-cultured with or without the test agent in the step (a) and further culturing them;
(c) measuring a degree of lymphocyte adhesion to the cells pre-cultured with the test agent, and comparing the measured degree with a degree of lymphocyte adhesion to the cells pre-cultured without a test agent, thereby determining whether the test agent inhibits lymphocyte adhesion; and
(d) administering the test agent determined to inhibit lymphocyte adhesion in the step (c) to an animal suffering from an inflammatory disease to examine a therapeutic effect.

30. A method for screening a medicament for inhibiting lymphocyte adhesion to an endothelial cell, which comprises the steps of:
(a) pre-culturing lymphocytes with or without a test agent;
(b) adding the lymphocytes pre-cultured with or without the test agent in the step (a) to cells expressing FEX-2 polypeptide and further culturing them; and
(c) measuring a degree of lymphocyte adhesion to the cells pre-cultured with the test agent, and comparing the measured degree with a degree of lymphocyte adhesion to the cells pre-cultured without the test agent, thereby determining whether the test agent inhibits lymphocyte adhesion.

31. A method for screening a medicament for preventing or treating an inflammatory disease, which comprises the steps of:
(a) pre-culturing lymphocytes with or without a test agent;
(b) adding the lymphocytes pre-cultured with or without the test agent in the step (a) to cells expressing FEX-2 polypeptide and further culturing them; and
(c) measuring a degree of lymphocyte adhesion to the cells pre-cultured with the test agent, and comparing the measured degree with a degree of lymphocyte adhesion to the cells pre-cultured without the test agent, thereby determining whether the test agent inhibits lymphocyte adhesion; and
(d) administering the test agent determined to inhibit lymphocyte adhesion in the step (c) to an animal suffering from an inflammatory disease to examine a therapeutic effect.


SEQUENCE LISTING (109 sequences)

SEQ ID NO: 1 - protein, 2551 aa, Homo sapiens
  PEPTIDE (1)..(2551): human FEX-2
MMLQHLVIFCLGLVVQNFCSPAETTGQARRCDRKSLLTIRTECRSCALNLGVKCPDGYTMITSGSVGVRDCRYTFEVRTYSLSLPGCRHICRKDYLQPRCCPGRWGPDCIECPGGAGSPCNGRGSCAEGMEGNGTCSCQEGFGGTACETCADDNLFGPSCSSVCNCVHGVCNSGLDGDGTCECYSAYTGPKCDKPIPECAALLCPENSRCSPSTEDENKLECKCLPNYRGDGKYCDPINPCLRKICHPHAHCTYLGPNRHSCTCQEGYRGDGQVCLPVDPCQINFGNCPTKSTVCKYDGPGQSHCECKEHYQNFVPGVGCSMTDICKSDNPCHRNANCTTVAPGRTECICQKGYVGDGLTCYGNIMERLRELNTEPRGKWQGRLTSFISLLDKAYAWPLSKLGPFTVLLPTDKGLKGFNVNELLVDNKAAQYFVKLHIIAGQMNIEYMNNTDMFYTLTGKSGEIFNSDKDNQIKLKLHGGKKKVKIIQGDIIASNGLLHILDRAMDKLEPTFESNNEQTIMTMLQPRYSKFRSLLEETNLGHALDEDGVGGPYTIFVPNNEALNNMKDGTLDYLLSPEGSRKLLELVRYHIVPFTQLEVATLISTPHIRSMANQLIQFNTTDNGQILANDVAMEEIEITAKNGRIYTLTGVLIPPSIVPILPHRCDETKREMKLGTCVSCSLVYWSRCPANSEPTALFTHRCVYSGRFGSLKSGCARYCNATVKIPKCCKGFYGPDCNQCPGGFSNPCSGNGQCADSLGGNGTCICEEGFQGSQCQFCSDPNKYGPRCNKKCLCVHGTCNNRIDSDGACLTGTCRDGSAGRLCDKQTSACGPYVQFCHIHATCEYSNGTASCICKAGYEGDGTLCSEMDPCTGLTPGGCSRNAECIKTGTGTHTCVCQQGWTGNGRDCSEINNCLLPSAGGCHDNASCLYVGPGQNECECKKGFRGNGIDCEPITSCLEQTGKCHPLASCQSTSSGVWSCVCQEGYEGDGFLCYGNAAVELSFLSEAAIFNRWINNASLQPTLSATSNLTVLVPSQQATEDMDQDEKSFWLSQSNIPALIKYHMLLGTYRVADLQTLSSSDMLATSLQGNFLHLAKVDGNITIEGASIVDGDNAATNGVIHIINKVLVPQRRLTGSLPNLLMRLEQMPDYSIFRGYIIQYNLANAIEAADAYTVFAPNNNAIENYIREKKVLSLEEDVLRYHVVLEEKLLKNDLHNGMHRETMLGFSYFLSFFLHNDQLYVNEAPINYTNVATDKGVIHGLGKVLEIQKNRCDNNDTTIIRGRCRTCSSELTCPFGTKSLGNEKRRCIYTSYFMGRRTLFIGCQPKCVRTVITRECCAGFFGPQCQPCPGNAQNVCFGNGICLDGVNGTGVCECGEGFSGTACETCTEGKYGIHCDQACSCVHGRCNQGPLGDGSCDCDVGWRGVHCDNATTEDNCNGTCHTSANCLTNSDGTASCKCAAGFQGNGTICTAINACEISNGGCSAKADCKRTTPGRRVCTCKAGYTGDGIVCLEINPCLENHGGCDKNAECTQTGPNQAACNCLPAYTGDGKVCTLINVCLTKNGGCSEFAICNHTGQVERTCTCKPNYIGDGFTCRGSIYQELPKNPKTSQYFFQLQEHFVKDLVGPGPFTVFAPLSAAFDEEARVKDWDKYGLMPQVLRYHVVACHQLLLENLKLISNATSLQGEPIVISVSQSTVYINNKAKIISSDIISTNGIVHIIDKLLSPKNLLITPKDNSGRILQNLTTLATNNGYIKFSNLIQDSGLLSVITDPIHTPVTLFWPTDQALHALPAEQQDFLFNQDNKDKLKEYLKFHVIRDAKVLAVDLPTSTAWKTLQGSELSVKCGAGRDIGDLFLNGQTCRIVQRELLFDLGVAYGIDCLLIDPTLGGRCDTFTTFDASGECGSCVNTPSCPRWSKPKGVKQKCLYNLPFKRNLEGCRERCSLVIQIPRCCKGYFGRDCQACPGGPDAPCNNRGVCLDQYSATGECKCNTGFNGTACEMCWPGRFGPDCLPCGCSDHGQCDDGITGSGQCLCETGWTGPSCDTQAVLPAVCTPPCSAHATCKENNTCECNLDYEGDGITCTVVDFCKQDNGGCAKVARCSQKGTKVSCSCQKGYKGDGHSCTEIDPCADGLNGGCHEHATCKMTGPGKHKCECKSHYVGDGLNCEPEQLPIDRCLQDNGQCHADAKCVDLHFQDTTVGVFHLRSPLGQYKLTFDKAREACANEAATMATYNQLSYAQKAKYHLCSAGWLETGRVAYPTAFASQNCGSGVVGIVDYGPRPNKSEMWDVFCYRMKDVNCTCKVGYVGDGFSCSGNLLQVLMSFPSLTNFLTEVLAYSNSSARGRAFLEHLTDLSIRGTLFVPQNSGLGENETLSGRDIEHHLANVSMFFYNDLVNGTTLQTRLGSKLLITASQDPLQPTETRFVDGRAILQWDIFASNGIIHVISRPLKAPPAPVTLTHTGLGAGIFFAIILVTGAVALAAYSYFRINRRTIGFQHFESEEDINVAALGKQQPENISNPLYESTTSAPPEPSYDPFTDSEERQLEGNDPLRTL

SEQ ID NO: 2 - protein, 103 aa, Homo sapiens
  DOMAIN (1)..(103): fas-1 domain of human FEX-2
TVLLPTDKGLKGFNVNELLVDNKAAQYFVKLHIIAGQMNIEYMNNTDMFYTLTGKSGEIFNSDKDNQIKLKLHGGKKKVKIIQGDIIASNGLLHILDRAMDKL

SEQ ID NO: 3 - protein, 102 aa, Homo sapiens
  DOMAIN (1)..(102): fas-1 domain of human FEX-2
TIFVPNNEALNNMKDGTLDYLLSPEGSRKLLELVRYHIVPFTQLEVATLISTPHIRSMANQLIQFNTTDNGQILANDVAMEEIEITAKNGRIYTLTGVLIPP

SEQ ID NO: 4 - protein, 101 aa, Homo sapiens
  DOMAIN (1)..(101): fas-1 domain of human FEX-2
TVLVPSQQATEDMDQDEKSFWLSQSNIPALIKYHMLLGTYRVADLQTLSSSDMLATSLQGNFLHLAKVDGNITIEGASIVDGDNAATNGVIHIINKVLVPQ

SEQ ID NO: 5 - protein, 96 aa, Homo sapiens
  DOMAIN (1)..(96): fas-1 domain of human FEX-2
TVFAPNNNAIENYIREKKVLSLEEDVLRYHVVLEEKLLKNDLHNGMHRETMLGFSYFLSFFLHNDQLYVNEAPINYTNVATDKGVIHGLGKVLEIQ

SEQ ID NO: 6 - protein, 97 aa, Homo sapiens
  DOMAIN (1)..(97): fas-1 domain of human FEX-2
TVFAPLSAAFDEEARVKDWDKYGLMPQVLRYHVVACHQLLLENLKLISNATSLQGEPIVISVSQSTVYINNKAKIISSDIISTNGIVHIIDKLLSPK

SEQ ID NO: 7 - protein, 106 aa, Homo sapiens
  DOMAIN (1)..(106): fas-1 domain of human FEX-2
TLFWPTDQALHALPAEQQDFLFNQDNKDKLKEYLKFHVIRDAKVLAVDLPTSTAWKTLQGSELSVKCGAGRDIGDLFLNGQTCRIVQRELLFDLGVAYGIDCLLID

SEQ ID NO: 8 - protein, 94 aa, Homo sapiens
  DOMAIN (1)..(94): fas-1 domain of human FEX-2
TLFVPQNSGLGENETLSGRDIEHHLANVSMFFYNDLVNGTTLQTRLGSKLLITASQDPLQPTETRFVDGRAILQWDIFASNGIIHVISRPLKAP

SEQ ID NO: 9 - protein, 590 aa, Homo sapiens
  PEPTIDE (1)..(590): Unit 1 of human FEX-2
VGVRDCRYTFEVRTYSLSLPGCRHICRKDYLQPRCCPGRWGPDCIECPGGAGSPCNGRGSCAEGMEGNGTCSCQEGFGGTACETCADDNLFGPSCSSVCNCVHGVCNSGLDGDGTCECYSAYTGPKCDKPIPECAALLCPENSRCSPSTEDENKLECKCLPNYRGDGKYCDPINPCLRKICHPHAHCTYLGPNRHSCTCQEGYRGDGQVCLPVDPCQINFGNCPTKSTVCKYDGPGQSHCECKEHYQNFVPGVGCSMTDICKSDNPCHRNANCTTVAPGRTECICQKGYVGDGLTCYGNIMERLRELNTEPRGKWQGRLTSFISLLDKAYAWPLSKLGPFTVLLPTDKGLKGFNVNELLVDNKAAQYFVKLHIIAGQMNIEYMNNTDMFYTLTGKSGEIFNSDKDNQIKLKLHGGKKKVKIIQGDIIASNGLLHILDRAMDKLEPTFESNNEQTIMTMLQPRYSKFRSLLEETNLGHALDEDGVGGPYTIFVPNNEALNNMKDGTLDYLLSPEGSRKLLELVRYHIVPFTQLEVATLISTPHIRSMANQLIQFNTTDNGQILANDVAMEEIEITAKNGRIYTLTGVLIPP

SEQ ID NO: 10 - protein, 578 aa, Homo sapiens
  PEPTIDE (1)..(578): Unit 2 of human FEX2
NSEPTALFTHRCVYSGRFGSLKSGCARYCNATVKIPKCCKGFYGPDCNQCPGGFSNPCSGNGQCADSLGGNGTCICEEGFQGSQCQFCSDPNKYGPRCNKKCLCVHGTCNNRIDSDGACLTGTCRDGSAGRLCDKQTSACGPYVQFCHIHATCEYSNGTASCICKAGYEGDGTLCSEMDPCTGLTPGGCSRNAECIKTGTGTHTCVCQQGWTGNGRDCSEINNCLLPSAGGCHDNASCLYVGPGQNECECKKGFRGNGIDCEPITSCLEQTGKCHPLASCQSTSSGVWSCVCQEGYEGDGFLCYGNAAVELSFLSEAAIFNRWINNASLQPTLSATSNLTVLVPSQQATEDMDQDEKSFWLSQSNIPALIKYHMLLGTYRVADLQTLSSSDMLATSLQGNFLHLAKVDGNITIEGASIVDGDNAATNGVIHIINKVLVPQRRLTGSLPNLLMRLEQMPDYSIFRGYIIQYNLANAIEAADAYTVFAPNNNAIENYIREKKVLSLEEDVLRYHVVLEEKLLKNDLHNGMHRETMLGFSYFLSFFLHNDQLYVNEAPINYTNVATDKGVIHGLGKVLEIQ

SEQ ID NO: 11 - protein, 581 aa, Homo sapiens
  PEPTIDE (1)..(581): Unit 3 of human FEX-2
EKRRCIYTSYFMGRRTLFIGCQPKCVRTVITRECCAGFFGPQCQPCPGNAQNVCFGNGICLDGVNGTGVCECGEGFSGTACETCTEGKYGIHCDQACSCVHGRCNQGPLGDGSCDCDVGWRGVHCDNATTEDNCNGTCHTSANCLTNSDGTASCKCAAGFQGNGTICTAINACEISNGGCSAKADCKRTTPGRRVCTCKAGYTGDGIVCLEINPCLENHGGCDKNAECTQTGPNQAACNCLPAYTGDGKVCTLINVCLTKNGGCSEFAICNHTGQVERTCTCKPNYIGDGFTCRGSIYQELPKNPKTSQYFFQLQEHFVKDLVGPGPFTVFAPLSAAFDEEARVKDWDKYGLMPQVLRYHVVACHQLLLENLKLISNATSLQGEPIVISVSQSTVYINNKAKIISSDIISTNGIVHIIDKLLSPKNLLITPKDNSGRILQNLTTLATNNGYIKFSNLIQDSGLLSVITDPIHTPVTLFWPTDQALHALPAEQQDFLFNQDNKDKLKEYLKFHVIRDAKVLAVDLPTSTAWKTLQGSELSVKCGAGRDIGDLFLNGQTCRIVQRELLFDLGVAYGIDCLLID

SEQ ID NO: 12 - protein, 537 aa, Homo sapiens
  PEPTIDE (1)..(537): Unit 4 of human FEX-2
RWSKPKGVKQKCLYNLPFKRNLEGCRERCSLVIQIPRCCKGYFGRDCQACPGGPDAPCNNRGVCLDQYSATGECKCNTGFNGTACEMCWPGRFGPDCLPCGCSDHGQCDDGITGSGQCLCETGWTGPSCDTQAVLPAVCTPPCSAHATCKENNTCECNLDYEGDGITCTVVDFCKQDNGGCAKVARCSQKGTKVSCSCQKGYKGDGHSCTEIDPCADGLNGGCHEHATCKMTGPGKHKCECKSHYVGDGLNCEPEQLPIDRCLQDNGQCHADAKCVDLHFQDTTVGVFHLRSPLGQYKLTFDKAREACANEAATMATYNQLSYAQKAKYHLCSAGWLETGRVAYPTAFASQNCGSGVVGIVDYGPRPNKSEMWDVFCYRMKDVNCTCKVGYVGDGFSCSGNLLQVLMSFPSLTNFLTEVLAYSNSSARGRAFLEHLTDLSIRGTLFVPQNSGLGENETLSGRDIEHHLANVSMFFYNDLVNGTTLQTRLGSKLLITASQDPLQPTETRFVDGRAILQWDIFASNGIIHVISRPLKAP

SEQ ID NO: 13 - protein, 96 aa, Mycobacterium tuberculosis
  DOMAIN (1)..(96): fas-1 domain of mpt70
TVFAPTNAAFSKLPASTIDELKTNSSLLTSILTYHVVAGQTSPANVVGTRQTLQGASVTVTGQGNSLKVGNADVVCGGVSTANATVYMIDSVLMPP

SEQ ID NO: 14 - protein, 96 aa, Mycobacterium tuberculosis
  DOMAIN (1)..(96): fas-1 domain of mpt83
TVFAPTNAAFDKLPAATIDQLKTDAKLLSSILTYHVIAGQASPSRIDGTHQTLQGADLTVIGARDDLMVNNAGLVCGGVHTANATVYMIDTVLMPP

SEQ ID NO: 15 - protein, 2559 aa, mouse
  PEPTIDE (1)..(2559): mouse FEX-2
MARSKLLLGKLLPLILIFLGLLVQNACSPTEAPELTKRCDKKSTLTIKTECQSCSVNIAVKCPDGYIKITNGTVGVRDCRYSLKIQSYVLDIPGCRHICRKDYLQPQCCPGHWGPDCMECPGGARAPCGGRGVCDEGMEGTGSCSCRAGFRGTACENCAAEDVFGPNCSAVCSCVHGVCNSGISGDGTCECLSAYRGPRCDKPIPECAALLCPENSRCSPSSKDETKLQCKCLPSYKGDGQTCKPINPCLKNVCHPHASCSYLGPNRHSCVCQKGYQGDGQVCLPVDPCQTSYGNCPTKSTVCRYDGPGQSHCECKEHYRNFVPGVGCSMTDICESKNPCHKNANCSTVSPGQTQCTCQKGYVGDGLNCYGNIMQRLRELNTEPRGMWQGQLTSFISILDRTYAWPLSNLGPFTVLLPSDKGLKGVDVKELLMDKEAARYFVKLHIIAGQMSTEQMYNLDTFYTLTGKSGEIINKDKDNQLKLKLYGSKIVQIIQGNIVASNGLVHILDRAMDKIEPTLESNPQQTIMTMLQPRYGKFRSLLEKTNVGQALEKGGIDEPYTIFVPSNEALSNMTAGVLDYLLSPEGSRKLLELVRYHIVAFTQLEVATLVSTLHIRSMANQIITFNISSKGQILANNVAVDETEVAAKNGRIYTLTGVLIPPSILPILPHRCNETKREMKLGTCVRCFMKNWSKCPTNSEPTAIFTNKCFYGSRAWNLKIGCARYCDVTVEIPRCCKGFFGPDCNPCPGGFMNPCSGNGQCIDGLGGNGTCICEDGFQGSRCQFCSKPNRYGPQCNRTCQCVHGICDNRLDSDGSCLPGTCREGTAGRFCDKQTSACGPYMQFCHIHATCEYSNETASCVCNDGYEGDGTLCSKKDPCLGSTSRGGCSPNAECIQASTGTYSCVCQRGWTGNGRDCVEINSCLLPSSGGCHDNATCLYVGPGQNECECKKGFRGNGIDCEPIISCLEQIEKCHPLATCQYTLSGVWSCVCQEGYEGNGVLCYGNVLMELSFLSEAAVFYQWINNASLQSMLSATSNLTVLVPSLQAIKDMDQNEKSFWLSRNNIPALIKYHTLLGTYRVADLQTLPSSHMLATSLQGSFLRLDKADGNITIEGASFVDGDNAATNGVVHIINKVLIPQRGLTGSLPSLLTRLEQMPDYSIFRGYIIHYNLASAIEAADAYTVFVPNNEAIESYIREKKATSLKEDILQYHVVLGEKLLRNDLHNGMHRETMLGFSYLLAFFLHNDQLYVNEAPINYTNVATDKGVIHGLEKVLEIKKNRCDNNDTIIVRGKCGKCSQQTLCPLETKPLSETRKCIYSVYFMGKRSIFIGCQLQCVRTIITSACCAGFFGPQCQACPGKGQNVCSGNGFCLDGVNGTGTCECEQGFNGTACETCTEGKYGIHCDQACSCVHGRCNQGPSGDGSCDCDVGWRGVKCDSEITTDNCNGTCHTSANCLLDPDGKASCKCAAGFQGNGTVCTAINACEISNGGCSAKADCKRTIPGSRVCVCKAGYTGDGIVCLEINPCLENHGGCDRHAECTQTGPNQAVCNCLPKYTGDGKVCTLINVCLTNNGGCSPFAFCNHTEQDQRTCTCKPDYTGDGIVCRGSIHSELPKNPSTSQYFFQLQEHAVQELAGPGPFTVFVPSSDSFNSESKLKVWDKQGLMSQILRYHVVACQQLLLENLKVITSATTLQGEPISISVSQDTVLINKKAKVLSSDIISTNGVIHVIDTLLSPQNLLITPKGASGRVLLNLTTVAANHGYTKFSKLIQDSGLLKVITDPMHTPVTLFWPTDKALQALPQEQQDFLFNEDNKDKLKAYLKFHVIRDTMALASDLPRSASWKTLQGSELSVRCGTGSDVGELFLNGQMCRIIQRRLLFDGGVAYGIDCLLMDPTEGGRCDTFTTFNIPGECGSCFSTPRCPLQSKPKGVRKKCIYNPLPFRRDVEGCQNLCTLVVHVPRCCSGYFMPDCQACPGGPDTPCNNRGMCYDQYKPTGQCQCHTGFNGTACELCLPGRFGPDCQPCGCSEHGQCDEGITGSGQCLCEAGWTGRFCDAPTVVIPVCIPACSMHATCMENNTCVCNLNYEGDGITCTVVDFCKQNNGGCAKVAKCSQKGTQVSCSCQKGYKGDGHSCTEIDPCANGVNGGCHEHATCRMTGPGKQKCECKSHYVGDGRDCEPEQLPLDRCLQDNGQCHPDANCVDLHFQDTTVGVFHLRSPLGQYKLTFDKAKEACAKEAASIATYNQLSYAQKAKYHLCSAGWLESGRVAYPTIYASKKCANIVGIVDYGTRTNKSEMWDVFCYRMKDVNCTCKAGYVGDGFSCNGNLLQVLMSFPSLTNFLTEVLVFSRSSAQGRAFLKHLTDLSISGTLFVPQNSGLPKNKSLSGRDIEHHLTNVNVSFYDDLVNGTVLKTRLGSQLLITSSQDQLHQEARFVDGRAILQWDIIASNGVLHIISEPLKAPPTAATAAHSGLGTGIFCAVVLVTGAIALAAYSYFRLNQRTTGFRRFESEDDIDALAFGKQQPESITNPLYETSTPAAPEPSCDPFTDSGERELENSDPLGALRS

SEQ ID NO: 16 - protein, 102 aa, mouse
  DOMAIN (1)..(102): fas-1 domain of mouse FEX-2
TVLLPSDKGLKGVDVKELLMDKEAARYFVKLHIIAGQMSTEQMYNLDTFYTLTGKSGEIINKDKDNQLKLKLYGSKIVQIIQGNIVASNGLVHILDRAMDKI

SEQ ID NO: 17 - protein, 102 aa, mouse
  DOMAIN (1)..(102): fas-1 domain of mouse FEX-2
TIFVPSNEALSNMTAGVLDYLLSPEGSRKLLELVRYHIVAFTQLEVATLVSTLHIRSMANQIITFNISSKGQILANNVAVDETEVAAKNGRIYTLTGVLIPP

SEQ ID NO: 18 - protein, 101 aa, mouse
  DOMAIN (1)..(101): fas-1 domain of mouse FEX-2
TVLVPSLQAIKDMDQNEKSFWLSRNNIPALIKYHTLLGTYRVADLQTLPSSHMLATSLQGSFLRLDKADGNITIEGASFVDGDNAATNGVVHIINKVLIPQ

SEQ ID NO: 19 - protein, 96 aa, mouse
  DOMAIN (1)..(96): fas-1 domain of mouse FEX-2
TVFVPNNEAIESYIREKKATSLKEDILQYHVVLGEKLLRNDLHNGMHRETMLGFSYLLAFFLHNDQLYVNEAPINYTNVATDKGVIHGLEKVLEIK

SEQ ID NO: 20 - protein, 97 aa, mouse
  DOMAIN (1)..(97): fas-1 domain of mouse FEX-2
TVFVPSSDSFNSESKLKVWDKQGLMSQILRYHVVACQQLLLENLKVITSATTLQGEPISISVSQDTVLINKKAKVLSSDIISTNGVIHVIDTLLSPQ

SEQ ID NO: 21 - protein, 107 aa, mouse
  DOMAIN (1)..(107): fas-1 domain of mouse FEX-2
TLFWPTDKALQALPQEQQDFLFNEDNKDKLKAYLKFHVIRDTMALASDLPRSASWKTLQGSELSVRCGTGSDVGELFLNGQMCRIIQRRLLFDGGVAYGIDCLLMDP

SEQ ID NO: 22 - protein, 96 aa, rat
  DOMAIN (1)..(96): fas-1 domain of rat FEX-2
TVFVPNNEAIENYIREKKATSLKEDILRYHVVLGEKLLKNDLHNGMHRETMLGFSYLLAFFLRNDQLYVNEAPINYTNVATDKGVIHGLEKVLEIQ

SEQ ID NO: 23 - protein, 97 aa, rat
  DOMAIN (1)..(97): fas-1 domain of rat FEX-2
TVFAPLSSSFNHEPRIKDWDQQGLMSQVLRYHVVGCQQLLLDNLKVTTSATTLQGEPVSISVSQDTVFINNEAKVLSSDIISTNGVIHVIDKLLSPK

SEQ ID NO: 24 - protein, 107 aa, rat
  DOMAIN (1)..(107): fas-1 domain of rat FEX-2
TVFWPTDKALEALPPEQQDFLFNQDNKDKLKSYLKFHVIRDSKALASDLPRSASWKTLQGSELSVRCGTGSDIGELFLNEQMCRFIHRGLLFDVGVAYGIDCLLMNP

SEQ ID NO: 25 - protein, 94 aa, rat
  DOMAIN (1)..(94): fas-1 domain of rat FEX-2
TLFVPQNSGLPGNKSLSGRDIEHHLTNVNVSFYNDLVNGTFLRTMLGSQLLITFSQDQLHQETRFVDGRSILQWDIIAANGILHIISEPLRAPP

SEQ ID NO: 26 - protein, 103 aa, Homo sapiens
  DOMAIN (1)..(103): fas-1 domain of human TGF-beta inducible gene-h3
GPGSFTIFAPSNEAWASLPAEVLDSLVSNVNIELLNALRYHMVGRRVLTDELKHGMTLTSMYQNSNIQIHHYPNGIVTVNCARLLKADHHATNGVVHLIDKVI

SEQ ID NO: 27 - protein, 122 aa, Homo sapiens
  DOMAIN (1)..(122): fas-1 domain of human TGF-beta inducible gene-h3
TFETLRAAVAASGLNTMLEGNGQYTLLAPTNEAFEKIPSETLNRILGDPEALRDLLNNHILKSAMCAEAIVAGLSVETLEGTTLEVGCSGDMLTINGKAIISNKDILATNGVIHYIDELLIP

SEQ ID NO: 28 - protein, 112 aa, Homo sapiens
  DOMAIN (1)..(112): fas-1 domain of human TGF-beta inducible gene-h3
STAIDLFRQAGLGNHLSGSERLTLLAPLNSVFKDGTPPIDAHTRNLLRNHIIKDQLASKYLYHGQTLETLGGKKLRVFVYRNSLCIENSCIAAHDKRGRYGTLFTMDRVLTP

SEQ ID NO: 29 - protein, 133 aa, Homo sapiens
  DOMAIN (1)..(133): fas-1 domain of human TGF-beta inducible gene-h3
MGTVMDVLKGDNRFSMLVAAIQSAGLTETLNREGVYTVFAPTNEAFRALPPRERSRLLGDAKELANILKYHIGDEILVSGGIGALVRLKSLQGDKLEVSLKNNVVSVNKEPVAEPDIMATNGVVHVITNVLQP

SEQ ID NO: 30 - protein, 103 aa, mouse
  DOMAIN (1)..(103): fas-1 domain of mouse TGF-beta inducible gene-h3
GPGSFTIFAPSNEAWSSLPAEVLDSLVSNVNIELLNALRYHMVDRRVLTDELKHGMTLTSMYQNSNIQIHHYPNGIVTVNCARLLKADHHATNGVVHLIDKVI

SEQ ID NO: 31 - protein, 122 aa, mouse
  DOMAIN (1)..(122): fas-1 domain of mouse TGF-beta inducible gene-h3
TFETLRAAVAASGLNTVLEGDGQFTLLAPTNEAFEKIPAETLNRILGDPEALRDLLNNHILKSAMCAEAIVAGMSMETLGGTTLEVGCSGDKLTINGKAVISNKDILATNGVIHFIDELLIP

SEQ ID NO: 32 - protein, 112 aa, mouse
  DOMAIN (1)..(112): fas-1 domain of mouse TGF-beta inducible gene-h3
STAIDILKQAGLDTHLSGKEQLTFLAPLNSVFKDGVPRIDAQMKTLLLNHMVKEQLASKYLYSGQTLDTLGGKKLRVFVYRNSLCIENSCIAAHDKRGRFGTLFTMDRMLTP

SEQ ID NO: 33 - protein, 133 aa, mouse
  DOMAIN (1)..(133): fas-1 domain of mouse TGF-beta inducible gene-h3
MGTVMDVLKGDNRFSMLVAAIQSAGLMEILNREGVYTVFAPTNEAFQAMPPEELNKLLANAKELTNILKYHIGDEILVSGGIGALVRLKSLQGDKLEVSSKNNVVSVNKEPVAETDIMATNGVVYAINTVLQP

SEQ ID NO: 34 - protein, 98 aa, rat
  DOMAIN (1)..(98): fas-1 domain of rat TGF-beta inducible gene-h3
HLSGKEQLTFLAPLNSVFKDGVPRIDGQMKTLLLNHMVKGQLASKYLYSGQTVDTLGGKKLRVFVYRNSLCIENSCIAAHDKKGRYGTLFTMDRMLTP

SEQ ID NO: 35 - protein, 111 aa, rat
  DOMAIN (1)..(111): fas-1 domain of rat TGF-beta inducible gene-h3
MGTVMDVLKGDNRFSMLVAAIQSAGLMETLNREGVYTVFAPTNEAFQAMPPEELNKLLANAKELTNILKYHIGDEILVSGGIGALVRLKSLQGDKLEVSSKNNVVSVNKEP

SEQ ID NO: 36 - protein, 120 aa, Homo sapiens
  DOMAIN (1)..(120): fas-1 domain of human periostin
TTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVL

SEQ ID NO: 37 - protein, 133 aa, Homo sapiens
  DOMAIN (1)..(133): fas-1 domain of human periostin
TSIQDFIEAEDDLSSFRAAAITSDILEALGRDGHFTLFAPTNEAFEKLPRGVLERFMGDKVASEALMKYHILNTLQCSESIMGGAVFETLEGNTIEIGCDGDSITVNGIKMVNKKDIVTNNGVIHLIDQVLIP

SEQ ID NO: 38 - protein, 116 aa, Homo sapiens
  DOMAIN (1)..(116): fas-1 domain of human periostin
KQQTTFTDLVAQLGLASALRPDGEYTLLAPVNNAFSDDTLSMVQRLLKLILQNHILKVKVGLNELYNGQILETIGGKQLRVFVYRTAVCIENSCMEKGSKQGRNGAIHIFREIIKP

SEQ ID NO: 39 - protein, 135 aa, Homo sapiens
  DOMAIN (1)..(135): fas-1 domain of human periostin
EKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYP

SEQ ID NO: 40 - protein, 120 aa, mouse
  DOMAIN (1)..(120): fas-1 domain of mouse periostin
TTTQHYSDVSKLREEIEGKGSYTYFAPSNEAWENLDSDIRRGLENNVNVELLNALHSHMVNKRMLTKDLKHGMVIPSMYNNLGLFINHYPNGVVTVNCARVIHGNQIATNGVVHVIDRVL

SEQ ID NO: 41 - protein, 133 aa, mouse
  DOMAIN (1)..(133): fas-1 domain of mouse periostin
TSIQDFLEAEDDLSSFRAAAITSDLLESLGRDGHFTLFAPTNEAFEKLPRGVLERIMGDKVASEALMKYHILNTLQCSEAITGGAVFETMEGNTIEIGCEGDSISINGIKMVNKKDIVTKNGVIHLIDEVLIP

SEQ ID NO: 42 - protein, 116 aa, mouse
  DOMAIN (1)..(116): fas-1 domain of mouse periostin
KQQTTFTDLVAQLGLASSLKPDGEYTLLAPVNNAFSDDTLSMDQRLLKLILQNHILKVKVGLSDLYNGQILETIGGKQLRVFVYRTAICIENSCMVRGSKQGRNGAIHIFREIIQP

SEQ ID NO: 43 - protein, 135 aa, mouse
  DOMAIN (1)..(135): fas-1 domain of mouse periostin
EKSLHDKLRQDKRFSIFLSLLEAADLKDLLTQPGDWTLFAPTNDAFKGMTSEERELLIGDKNALQNIILYHLTPGVYIGKGFEPGVTNILKTTQGSKIYLKGVNETLLVNELKSKESDIMTTNGVIHVVDKLLYP

SEQ ID NO: 44 - protein, 120 aa, rat
  DOMAIN (1)..(120): fas-1 domain of rat periostin
TTTQHYSDVSKLREEIEGKGSYTYFAPSNEAWDNLDSDIRRGLENNVNVELLNALHSHMVNKRMLTKDLKHGMVIPSMYNNLGLFINHYPNGVVTVNCARVIHGNQIATNGVVHVIDRVL

SEQ ID NO: 45 - protein, 133 aa, rat
  DOMAIN (1)..(133): fas-1 domain of rat periostin
TSIQDFIEAEDELSSFRAAAITSDLLESLGRDGHFTLFAPTNEAFEKLPRGVLERIMGDKVASEALMKYHILNTLQCSEAITGGAVFETMEGNTIEIGCEGDSISINGIKMVNKKDIVTKNGVIHLIDEVLIP

SEQ ID NO: 46 - protein, 116 aa, rat; identical to SEQ ID NO: 42
  DOMAIN (1)..(116): fas-1 domain of rat periostin

SEQ ID NO: 47 - protein, 135 aa, rat
  DOMAIN (1)..(135): fas-1 domain of rat periostin
EKSLHEKLRQDKRFSIFLSLLEAADLKDLLTQPGDWTLFAPTNDAFKGMTNEEREILIGDKNALQNIILYHLTPGVYIGKGFEPGVTNILKTTQGSKIYVKGVNETLLVNELKSKESDIMTTNGVIHVVDKLLYP

SEQ ID NO: 48 - protein, 101 aa, Homo sapiens
  DOMAIN (1)..(101): fas-1 domain of human FEX-1
TVLVPSVSSFSSRTMNASLAQQLCRQHIIAGQHILEDTRTQQTRRWWTLAGQEITVTFNQFTKYSYKYKDQPQQTFNIYKANNIAANGVFHVVTGLRWQAP

SEQ ID NO: 49 - protein, 104 aa, Homo sapiens
  DOMAIN (1)..(104): fas-1 domain of human FEX-1
FTVFAPSNEAVDSLRDGRLIYLFTAGLSKLQELVRYHIYNHGQLTVEKLISKGRILTMANQVLAVNISEEGRILLGPEGVPLQRVDVMAANGVIHMLDGILLPP

SEQ ID NO: 50 - protein, 100 aa, Homo sapiens
  DOMAIN (1)..(100): fas-1 domain of human FEX-1
TALVPSEAAVRQLSPEDRAFWLQPRTLPNLVRAHFLQGALFEEELARLGGQEVATLNPTTRWEIRNISGRVWVQNASVDVADLLATNGVLHILSQVLLPP

SEQ ID NO: 51 - protein, 93 aa, Homo sapiens
  DOMAIN (1)..(93): fas-1 domain of human FEX-1
TIFVPTNRSLEAQGNSSHLDADTVRHHVVLGEALSMETLRKGGHRNSLLGPAHWIVFYNHSGQPEVNHVPLEGPMLEAPGRSLIGLSGVLTVG

SEQ ID NO: 52 - protein, 97 aa, Homo sapiens
  DOMAIN (1)..(97): fas-1 domain of human FEX-1
TIFVPHADLMSNLSQDELARIRAHRQLVFRYHVVGCRRLRSEDLLEQGYATALSGHPLRFSEREGSIYLNDFARVVSSDHEAVNGILHFIDRVLLPP

SEQ ID NO: 53 - protein, 106 aa, Homo sapiens
  DOMAIN (1)..(106): fas-1 domain of human FEX-1
TMLWPTDAAFRALPPDRQAWLYHEDHRDKLAAILRGHMIRNVEALASDLPNLGPLRTMHGTPISFSCSRTRPGELMVGEDDARIVQRHLPFEGGLAYGIDQLLEPP

SEQ ID NO: 54 - protein, 96 aa, Homo sapiens
  DOMAIN (1)..(96): fas-1 domain of human FEX-1
TLFVPVNEGFVDNMTLSGPDLELHASNATLLSANASQGKLLPAHSGLSLIISDAGPDNSSWAPVAPGTVVVSRIIVWDIMAFNGIIHALASPLLAP

SEQ ID NO: 55 - protein, 103 aa, mouse
  DOMAIN (1)..(103): fas-1 domain of mouse FEX-1
TVFAPSNEAVDSLRDGRLIYLFTAGLSKLQELVRYHIYNHGQLTVEKLISKGRVLTMANQVLTVNISEGGRILLGPGGIPVRRVDVPAANGVIHMLEGILLPP

SEQ ID NO: 56 - protein, 100 aa, mouse
  DOMAIN (1)..(100): fas-1 domain of mouse FEX-1
TALVPSESAIRRLSLEDQAFWLQPKMLPELARAHFLQGAFSEEELARLNGQQVATLSATTRWQIHNISGKVWVQNATVDVPDLLATNGILHIVSQVLLPP

SEQ ID NO: 57 - protein, 93 aa, mouse
  DOMAIN (1)..(93): fas-1 domain of mouse FEX-1
TIFVPTNHSLETQGNNSVLGIDTVRHHVILGEALSVEVLRKGGHRNSLLGPAHWLVFYNHSGQPEVNHMPLEGPLLEAPGSSLFGLSGILAVG

SEQ ID NO: 58 - protein, 97 aa, mouse
  DOMAIN (1)..(97): fas-1 domain of mouse FEX-1
TVFVPHADLISNMSQDELARIRAHRQLVFRYHVVGCRKLWSQEMLDQGYITTLSGHTLRVSEREGSIYLNDFARVVSSDLEVVNGVLHFIDHVLLPP

SEQ ID NO: 59 - protein, 106 aa, mouse
  DOMAIN (1)..(106): fas-1 domain of mouse FEX-1
TMLWPTDSALQALPPDRKNWLFHEDHRDKLAAILRGHMIRNIEALASDLPNLGQLRTMHGNTISFSCGLTRPGELIVGEDEAHIVQRHLTFEGGLAYGIDQLLEPP

SEQ ID NO: 60 - protein, 96 aa, mouse
  DOMAIN (1)..(96): fas-1 domain of mouse FEX-1
TLFVPVNKGFVDNMTLSGPDLELHASNATFLSINASRGTLLPAHSGLSLFISDTGPDNTSLVPLAPGAVVVSHVIVWDIMAFNGIIHALASPLLMP

SEQ ID NO: 61 - protein, 103 aa, rat; identical to SEQ ID NO: 55
  DOMAIN (1)..(103): fas-1 domain of rat FEX-1

SEQ ID NO: 62 - protein, 100 aa, rat; identical to SEQ ID NO: 56
  DOMAIN (1)..(100): fas-1 domain of rat FEX-1

SEQ ID NO: 63 - protein, 93 aa, rat; identical to SEQ ID NO: 57
  DOMAIN (1)..(93): fas-1 domain of rat FEX-1

SEQ ID NO: 64 - protein, 97 aa, rat; identical to SEQ ID NO: 58
  DOMAIN (1)..(97): fas-1 domain of rat FEX-1

SEQ ID NO: 65 - protein, 106 aa, rat; identical to SEQ ID NO: 59
  DOMAIN (1)..(106): fas-1 domain of rat FEX-1

SEQ ID NO: 66 - protein, 96 aa, rat; identical to SEQ ID NO: 60
  DOMAIN (1)..(96): fas-1 domain of rat FEX-1

SEQ ID NO: 67 - DNA, 22 bp, Artificial Sequence
  Fex23 sense primer
tgccccatcgatgtgatgaaac

SEQ ID NO: 68 - DNA, 25 bp, Artificial Sequence
  Fex23 antisens primer
caaacaagagctcccgctgcacaat

SEQ ID NO: 69 - DNA, 25 bp, Artificial Sequence
  Fex45 sense primer
gcagcgggagctcttgtttgacctg

SEQ ID NO: 70 - DNA, 25 bp, Artificial Sequence
  Fex45 antisens primer
cccgtccaagcttgcacagtgtcct

SEQ ID NO: 71 - DNA, 24 bp, Artificial Sequence
  RACE-GSP-1 antisense primer
tcccagcttactcagtggccaggc

SEQ ID NO: 72 - DNA, 29 bp, Artificial Sequence
  RACE-GSP-2 antisense primer
caggcccatcatatttgcacactgtagac

SEQ ID NO: 73 - DNA, 26 bp, Artificial Sequence
  RACE-GSP-3 antisense primer
agttaatttggcaggggtccacaggc

SEQ ID NO: 74 - DNA, 25 bp, Artificial Sequence
  FEX1 sense primer
aaggcaggtctcacctatctcctgg

SEQ ID NO: 75 - DNA, 22 bp, Artificial Sequence
  FEX1 antisense primer
tcacaaatgcatgtcccgttgc

SEQ ID NO: 76 - DNA, 25 bp, Artificial Sequence
  hFex2-5 sense primer
caaatgtgtcgacctccacttccag

SEQ ID NO: 77 - DNA, 25 bp, Artificial Sequence
  hFex2-5 antisense primer
cccgtccaagcttgcacagtgtcct

SEQ ID NO: 78 - DNA, 26 bp, Artificial Sequence
  hFex2-2 sense primer
ataaggatccaccatttttgttccaa

SEQ ID NO: 79 - DNA, 26 bp, Artificial Sequence
  hFex2-2 antisense primer
aataactcgagactgggaatgagaac

SEQ ID NO: 80 - DNA, 25 bp, Artificial Sequence
  hFex2-4E5 sense primer
aaaaaggatccacagtgtttgctcc

SEQ ID NO: 81 - DNA, 27 bp, Artificial Sequence
  hFex2-4E5 antisense primer
ttttactcgagacttttgggagatagc

SEQ ID NO: 82 - DNA, 28 bp, Artificial Sequence
  Nus-U1 sense primer
aaaaaggatccgtaggggttcgagattg

SEQ ID NO: 83 - DNA, 29 bp, Artificial Sequence
  Nus-U1 antisense primer
aataactcgagactgggaggaatgagaac

SEQ ID NO: 84 - DNA, 27 bp, Artificial Sequence
  Nus-U2 sense primer
aaaaaggatccaactctgagcccacag

SEQ ID NO: 85 - DNA, 36 bp, Artificial Sequence
  Nus-U2 antisene primer
aaatactcgagtgactgaatttccagaacttttccc

SEQ ID NO: 86 - DNA, 26 bp, Artificial Sequence
  Nus-U3 sense primer
aaaaaggatccgagaagaggagatgc

SEQ ID NO: 87 - DNA, 28 bp, Artificial Sequence
  Nus-U3 antisense primer
ttttactcgagactatcaatcagcagac

SEQ ID NO: 88 - DNA, 29 bp, Artificial Sequence
  Nus-U4 sense primer
aaaatggatccaggtggagtaaaccaaag

SEQ ID NO: 89 - DNA, 30 bp, Artificial Sequence
  Nus-U4 antisense primer
aaatagaattctcagggtgcttttaaaggc

SEQ ID NO: 90 - DNA, 26 bp, Artificial Sequence
  Nus-EGF3 sense primer
aaaaaggatccgagaagaggagatgc

SEQ ID NO: 91 - DNA, 29 bp, Artificial Sequence
  Nus-EGF3 antisense primer
aaaaactcgagtcaggatttacctgccgc

SEQ ID NO: 92 - DNA, 25 bp, Artificial Sequence
  Nus-Fas5 sense primer
ttttaggatccactgtttttgcacc

SEQ ID NO: 93 - DNA, 27 bp, Artificial Sequence
  Nus-Fas5 antisense primer
ttttactcgagacttttgggagatagc

SEQ ID NO: 94 - DNA, 25 bp, Artificial Sequence
  Nus-Fas6 sense primer
ttttaggatccactctcttctggcc

SEQ ID NO: 95 - DNA, 28 bp, Artificial Sequence
  Nus-Fas6 antisens primer
ttttactcgagactatcaatcagcagac

SEQ ID NO: 96 - DNA, 25 bp, Artificial Sequence
  Nus-Fas1 sense primer
aaaaaggatccacggtgctgttacc

SEQ ID NO: 97 - DNA, 31 bp, Artificial Sequence
  Nus-Fas1 antisense primer
aaaaactcgagacttaacttgtccatggctc

SEQ ID NO: 98 - DNA, 26 bp, Artificial Sequence
  Nus-Fas2 sense primer
ataaggatccaccatttttgttccaa

SEQ ID NO: 99 - DNA, 29 bp, Artificial Sequence
  Nus-Fas2 antisense primer
aataacycgagactgggaggaatgagaac

SEQ ID NO: 100 - DNA, 28 bp, Artificial Sequence
  Nus-Fas3 sense primer
tttacggatccactgtcctcgtgccttc

SEQ ID NO: 101 - DNA, 27 bp, Artificial Sequence
  Nus-Fas3 antisense primer
tttaactcgagactttgtgggaccagc

SEQ ID NO: 102 - DNA, 25 bp, Artificial Sequence
  Nus-Fas4 sense primer
aaaaaggatccacagtgtttgctcc

SEQ ID NO: 103 - DNA, 36 bp, Artificial Sequence; identical to SEQ ID NO: 85
  Nus-Fas4 antisense primer

SEQ ID NO: 104 - DNA, 27 bp, Artificial Sequence
  Nus-Fas7 sense primer
tttaaggatccaccctctttgtgccac

SEQ ID NO: 105 - DNA, 30 bp, Artificial Sequence; identical to SEQ ID NO: 89
  Nus-Fas7 antisense primer

SEQ ID NO: 106 - DNA, 24 bp, Artificial Sequence
  Nus-mpt70 sense primer
aaaaaggatccacggtgttcgcac

SEQ ID NO: 107 - DNA, 27 bp, Artificial Sequence
  Nus-mpt70 antisense primer
aaaatctcgagtcacggaggcattagc

SEQ ID NO: 108 - DNA, 24 bp, Artificial Sequence
  Nus-mpt83 sense primer
aattaggatccaccgttttcgccc

SEQ ID NO: 109 - DNA, 26 bp, Artificial Sequence
  Nus-mpt83 antisense primer
aaaatctcgagtcacgggggcatcag